(19) United States
(12) Patent Application Publication
(10) Pub. No.: US 2019/0030151 A1
(43) Pub. Date: Jan. 31, 2019

(54) METHODS AND COMPOSITIONS FOR T-CELL IMMUNOTHERAPY

(71) Applicant: ETUBICS CORPORATION, Seattle, WA (US)

(72) Inventors: Frank R. Jones, Seattle, WA (US); Elizabeth Gabitzsch, Seattle, WA (US); Yvette Latchman, Seattle, WA (US); Adrian Rice, Seattle, WA (US)

(21) Appl. No.: 16/070,201

(22) PCT Filed: Jan. 13, 2017
(86) PCT No.: PCT/US2017/013455

§ 371 Date: Jul. 13, 2018

Related U.S. Application Data
(60) Provisional application No. 62/279,275, filed on Jan. 15, 2016.

(51) Int. Cl. A61K 39/00; C12N 5/0783; C07K 16/30; C07K 14/705; C07K 14/725; A61K 45/06; A61P 35/00

(52) U.S. Cl. CPC A61K 39/001182; C12N 5/0636; C07K 16/3007; C07K 14/70521; A61K 2039/5156; A61K 45/06; A61P 35/00; C12N 2510/00; C07K 2319/02; C07K 14/7051

(57) ABSTRACT

Genetically modified compositions, such as adenoviral vectors and T cells, for treating diseases such as cancer and infectious diseases are disclosed. Also disclosed are the methods of making and using the genetically modified compositions in treating diseases such as cancer and infectious diseases.

### CROSS REFERENCE

This application claims the benefit of U.S. Provisional Patent Application No. 62/279,275, filed Jan. 15, 2016, the entire content of which is incorporated by reference.

### FIELD OF THE INVENTION

The invention relates to T cell-based immunotherapeutics and methods of using the therapeutics in immunotherapy, such as in the treatment of cancer.

### BACKGROUND

The use of T cells as a therapeutic agent against cancer is based on the premise that tumor-specific cells can be generated and expanded ex vivo and reinfused into a patient, Wang, X and Riviere, I, Cancer Gene Ther., 2015, 22, 85-94. Chimeric antigen receptors (CARs) are recombinant receptors for antigen, which, in a single molecule, redirect the specificity and function of T cells and other immune cells. The general premise for their use in cancer immunotherapy is to rapidly generate tumor-targeted T cells, bypassing the obstacles of active immunization. Once expressed in cells, the CAR-modified T cell can exert both immediate and long-term effects in a patient.

Retroviruses and lentiviruses remain the principle mechanism of CAR transduction in T cells. Despite the severe complications related to insertional mutagenesis in stem cells observed in early gene therapy trials, retroviruses have been widely used in terminally differentiated T cells without such complications. However, initial data suggest that less mature or central memory T-cells are more likely to proliferate and persist longer in patients compared to their more differentiated counterparts. Unlike retroviruses and lentiviruses, the adenoviral genome is maintained extra-chromosomally, therefore bypassing insertion site-dependent effects of the host genome, and extending the benefits of adenoviral transduction to T cell therapy platforms. The object of the present invention addresses the need for safely delivering a transgene without the risk of insertional mutagenesis to primary cells.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

In various aspects, the present disclosure provides a cell comprising: (a) at least one engineered receptor; and (b) at least one extra-chromosomal adenoviral genome; wherein the adenoviral genome has at least one deletion in a region of an adenoviral gene and encodes the engineered receptor.

In some aspects, the engineered receptor is a chimeric antigen receptor (CAR), a T-cell receptor (TCR), or a B-cell receptor (BCR), or a derivative thereof. In other aspects, the engineered receptor is a chimeric antigen receptor (CAR). In some aspects, the CAR is a first generation CAR. In other aspects, the CAR is a second generation CAR. In still other aspects, the CAR is a third generation CAR.

In some aspects, the CAR comprises an extracellular portion, a transmembrane portion, and an intracellular portion. In some aspects, the intracellular portion comprises at least one T cell co-stimulatory domain. In some aspects, the T cell co-stimulatory domain is selected from the group consisting of CD27, CD28, TNFRS9 (4-1BB), TNFRSF4 (OX40), TNFRSF8 (CD30), CD40LG (CD40L), ICOS, ITGB2 (LFA-1), CD2, CD7, KLRC2 (NKG2C), TNFRS18 (GITR), TNFRSF14 (HVEM), or any combination thereof.

In some aspects, the engineered receptor binds a target. In some aspects, the binding is MHC independent.

In other aspects, the binding is MHC dependent. In some aspects, the binding is specific to a disease-associated target. In some aspects, the disease is cancer. In further aspects, the cancer is a solid tumor. In other aspects, the cancer is a liquid tumor.

In some aspects, the receptor binds a target antigen. In some aspects, the target antigen is a tumor cell neo-antigen, a tumor neo-epitope, tumor-specific antigen, a tumor associated antigen, a tissue-specific antigen, a bacterial antigen, a viral antigen, a yeast antigen, a fungal antigen, a protozoan antigen, a parasite antigen, a mitogen, or a combination thereof.

In further aspects, the target antigen is selected from the group consisting of carcinoembryonic antigen (CEA), human epidermal growth factor receptor 1 (HER1) human epidermal growth factor receptor 2 (HER2/neu), human epidermal growth factor receptor 3 (HER3), human epidermal growth factor receptor 4 (HER4), human papillomavirus (HPV), mucin 1 (MUC1), prostate-specific antigen (PSA), PSMA, Brachyury, folate receptor alpha, WT1, p53, MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MC1R, Gp100, Tyrosinase, TRP-1, TRP-2, ART-4, CAMEL, Cyp-B, BRCA1, BRACHYURY(TIVS7-2, polymorphism), BRACHYURY (IVS7 T/C polymorphism), T BRACHYURY, T, hTERT, hTRT, iCE, MUC1 (VNTR polymorphism), MUC1c, MUC1n, MUC2, PRAME, P15, RU1, RU2, SART-1, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, or TEL/AML1, or a modified variant, a splice variant, a functional epitope, an epitope agonist,or a combination thereof.

In some aspects, the receptor binds a tumor-associated cell. In some aspects, the the tumor-associated cell is selected from the group consisting of a fibroblast, a cancer-stem cell, a pericyte, and a stromal cell.

In further aspects, the cell further comprises a secondary receptor. In some aspects, the secondary receptor is a coxsackie adenoviral receptor. In some aspects, the extra-chromosomal adenoviral genome is adenovirus serotype 5 (Ad5).

In some aspects, the deletion in a region of an adenoviral gene is a deletion in a region of an early region 1 (E1) gene, a deletion in an early region 2b (E2b) gene, a deletion in an early region 3 (E3) gene, or a combination thereof. In some aspects, the deletion in a region of an adenovirus gene is a deletion in a region of an early region 2b (E2b) gene. In some aspects, the deletion in a region of an adenoviral gene is a deletion in an early region 1 (E1) gene, an early region 2b (E2b) gene, and early region 3 (E3) gene.

In further aspects, the cell further comprises an exogenous gene. In some aspects, the exogenous gene is selected from a list comprising a suicide gene, a cytokine gene, an anti-angiogenic gene, a metabolism gene, or a hypoxia gene. In some aspects, the cell further comprises an endogenous gene deletion.

In some aspects, the cell is an immune cell. In some aspects, the immune cell is a T cell. In further aspects, the T cell is an effector (T_{EFF}) cell, effector-memory (T_{EM}) cell, central-memory (T_{CM}), T memory stem (T_{SCM}), naive (T_{N}), or CD4+ or CD8+. In some aspects, the cell is a primate cell. In further aspects, the cell is a human cell.

In some aspects, the cell is expanded ex vivo. In some aspects, the cell is formulated into a pharmaceutical composition. In some aspects, the cell is part of a combination therapy to treat a subject in need thereof. In some aspects, the engineered receptor is integrated into the genome of the subject in need thereof.

In various aspects, the present disclosure provides a method of preparing a cell, comprising contacting a cell ex vivo with at least one engineered extrachromosomal vector comprising at least one exogenous receptor sequence. In some aspects, the extrachromosomal vector is an adenoviral vector. In some aspects, the adenoviral vector is adenovirus serotype 5 (Ad5). In some aspects, the vector has at least one gene deletion.

In some aspects, the deletion is a deletion in a region of an early region 1 (E1) gene, and an early region 3 (E3) gene. In some aspects, the deletion is a deletion in an early region 2b (E2b) gene, a deletion in an early region 3 (E3) gene, or a combination thereof. In other aspects, the vector contains deletions in an early region 1 (E1) gene, an early region 2b (E2b) gene, and early region3 (E3) gene.

In some aspects, the vector is not a gutted vector. In further aspects, the method further comprises introducing at least one secondary receptor before the exogenous receptor. In some aspects, the secondary receptor is coxsackie adenovirus receptor. In some asepcts, the exogenous receptor sequence is selected from a list comprising a chimeric antigen receptor (CAR), a T-cell receptor (TCR), or a B-cell receptor (BCR), or a derivative thereof.

In other aspects, the exogenous receptor sequence encodes a chimeric antigen receptor (CAR). In some aspects, the vector further comprises a second exogenous gene sequence. In some aspects, the exogenous gene sequence is selected the group consisting of a suicide gene, a cytokine gene, an anti-angiogenic gene, a metabolism gene, and a hypoxia gene.

In some aspects, the second exogenous gene sequence comprises an inducible suicide gene sequence. In further aspects, the inducible suicide gene sequence is an inducible caspase 9 gene sequence or a portion of the EGF receptor R sequence.

In some aspects, the exogenous receptor sequence is introduced into the cell with at least one vector. In some aspects, the cell is activated ex vivo. In other aspects, the activation occurs before the exogenous receptor sequence is introduced. In some aspects, the activation is performed with anti-CD3 (OKT3), anti-CD28, at least one cytokine, or any combination thereof.

In further aspects, the cytokine comprises interleukin-2 (IL-2), interleukin-7 (IL-7), interleukin-15 (IL-15), interleukin-21 (IL-21), or any combination thereof.

In some aspects, the method further comprises expanding the cell. In some aspects, the cell is autologous to a subject in need thereof. In other aspects, the cell is allogenic to a subject in need thereof. In some aspects, the subject in need thereof has preexisting immunity to the adenoviral vector.

In some aspects, the cell is a good manufacturing practices (GMP) compatible reagent. In some aspects, the reagent is part of a combination therapy to treat cancer.

In various aspects, the present disclosure provides a pharmaceutical composition comprising the cell of any one of the above descriptions or a cell prepared according to any one of the methods described above.

In various aspects, the present disclosure provides a method of treating a condition in a subject in need thereof comprising administering to a subject a therapeutically effective amount of the pharmaceutical composition described above. In some aspects, the pharmaceutical composition is administered intravenously. In other aspects, the pharmaceutical composition is administered locally to a tumor. In some aspects, the method further comprises administering one or more additional therapeutic agents or treating the subject with one or more additional therapies.

In some aspects, the treating the subject with one or more additional therapies comprises transplantation. In other aspects, the treating the subject with one or more additional therapies comprises immunotherapy. In some aspects, the pharmaceutical composition is autologous to the subject. In other aspects, the pharmaceutical composition is allogenic to the subject.

In some aspects, the method further comprises administering to the subject a pharmaceutical composition comprising a population of engineered nature killer (NK) cells. In some aspects, the engineered NK cells comprise one or more NK cells that have been modified as essentially lacking the expression of MR (killer inhibitory receptors), one or more NK cells that have been modified to express a high affinity CD16 variant, and one or more NK cells that have been modified to express one or more CARs (chimeric antigen receptors), or any combinations thereof. In some aspects, the engineered NK cells comprise one or more NK cells that have been modified as essentially lacking the expression KIR.

In other aspects, the engineered NK cells comprise one or more NK cells that have been modified to express a high affinity CD16 variant. In some aspects, the engineered NK cells comprise one or more NK cells that have been modified to express one or more CARs. In some aspects, the CAR is a CAR for a tumor neo-antigen, tumor neo-epitope, HPV, PSA, PSMA, WT1, p53, MAGE-A1, MAGE-A2, MAGE-A3, MAGE-A4, MAGE-A6, MAGE-A10, MAGE-A12, BAGE, DAM-6, DAM-10, Folate receptor alpha, GAGE-1, GAGE-2, GAGE-8, GAGE-3, GAGE-4, GAGE-5, GAGE-6, GAGE-7B, NA88-A, NY-ESO-1, MART-1, MC1R, Gp100, Tyrosinase, TRP-1, TRP-2, ART-4, CAMEL, CEA, Cyp-B, HER1, HER2/neu, HERS, HER4, BRCA1, Brachyury, Brachyury (TIVS7-2, polymorphism), Brachyury (IVS7 T/C polymorphism), T Brachyury, T, hTERT, hTRT, iCE, MUC1, MUC1 (VNTR polymorphism), MUC1c, MUC1n, MUC2, PRAME, P15, RU1, RU2, SART-1, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPl/mbcr-abl, ETV6/AML, LDLR/FUT, Pml/RARα, TEL/AML1, or any combination thereof.

Embodiments discussed in the context of methods and/or compositions described herein can be employed with respect to any other method or composition described herein. Thus, an embodiment pertaining to one method or composition can be applied to other methods and compositions as well.

Other objects, features and advantages will become apparent from the following detailed description. It should be understood, however, that the detailed description and the specific examples, while indicating particular embodiments, are given by way of illustration only, since various changes and modifications within the spirit and scope of the invention will become apparent to those skilled in the art from this detailed description.

### BRIEF DESCRIPTION OF THE DRAWINGS

FIG. 1 illustrates schematics for the proposed manufacture and production of chimeric antigen receptor (CAR) T Cells and CAR Natural Killer (NK) cells.

FIG. 1A illustrates a schematic for the proposed manufacture and production of CAR T cells using the Ad5 [E1-, E2b-] platform. T cells are selected from washed apheresis collected patient product and activated using CD3/28 Dynabeads and ClinExVivo magnetic particle concentrator (MPC). Activated T cells are transduced with a CAR Ad5 [E1-, E2b-]-based vector. Transduced CAR T cells are expanded in a cell bioreactor. The CD3/28 Dynabeads are removed from cells using the MPC. At the end of the process, CAR T cells are formulated for infusion.

FIG. 1B illustrates a schematic for the propose manufacture and production of CAR NK cells using the Ad5 [E1-, E2b-] platform. Activated NK cells are transduced with a CAR Ad5 [E1-, E2b-]-based vector. Transduced CAR-NK cells are expanded in a cell bioreactor. At the end of the process, CAR-NK cells are formulated for infusion.

FIG. 2 illustrates a representation of CAR T cell expression after transfection with CAR based Ad5 [E1-, E2b-] platform chimeric antigen receptors (CARs) are composed of three regions, each displaying a function. The extracellular region of the CAR is generally composed of a single chain variable fragment (scFv). Its target is derived from fused variable heavy and light chains of a specifically constructed antibody. The transmembrane region of the CAR is connected to the scFv through a “spacer” and provides flexibility and stable expression of the extracellular antibody moiety. An intracellular signaling domain of the CAR, usually derived from the CD3ζ-chain of the T cell receptor (TCR)-CD3 complex, mediates activation of CAR T cells.

FIG. 3 illustrates schematic representations of various CAR vectors for insertion into the Ad5 [E1-, E2b-] platform.

FIG. 3A illustrates a first generation CAR containing a CD3-zeta signalling domain.

FIG. 3B illustrates two possible second generation CARs encoding for CD28 and CD3 zeta signalling domains and a second vector encoding for 4-1BB and CD3 zeta signalling domains.

FIG. 3C illustrates two possible third generation CARs encoding for CD28, 4-1BB, and CD3 zeta and a second CAR encoding for CD28, OX40, and CD3 zeta.

FIG. 3D illustrates a multicistronic CAR encoding for a caspase-9 suicide gene and a third generation CAR.

### DETAILED DESCRIPTION

As used herein, unless otherwise indicated, the article “a” means one or more unless explicitly otherwise provided for. As used herein, unless otherwise indicated, terms such as “contain,” “containing,” “include,” “including,” and the like mean “comprising.” As used herein, unless otherwise indicated, the term “or” can be conjunctive or disjunctive. As used herein, unless otherwise indicated, any embodiment can be combined with any other embodiment. As used herein, the phrase “at least one” can mean “at least one” or “a plurality.”

As used herein, unless otherwise indicated, some inventive embodiments herein contemplate numerical ranges. A variety of aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges include the range endpoints or any ranges derived from.

The term “activation” and its grammatical equivalents as used herein can refer to a process whereby a cell transitions from a resting state to an active state. This process can comprise a response to an antigen, migration, and/or a phenotypic or genetic change to a functionally active state. For example, the term “activation” can refer to the stepwise process of T cell activation. For example, a T cell can require at least two signals to become fully activated. The first signal can occur after engagement of a TCR by the antigen-MHC complex, and the second signal can occur by engagement of co-stimulatory molecules (e.g., a co-stimulatory molecule shown in TABLE 2). Anti-CD3 can mimic the first signal and anti-CD28 can mimic the second signal in vitro. For example, an engineered T cell can be activated by an expressed CAR. T cell activation” or T cell triggering, as used herein, can refer to the state of a T cell that has been sufficiently stimulated to induce detectable cellular proliferation, cytokine production and/or detectable effector function.

The term “adenovirus” or “Ad” refers to a group of non-enveloped DNA viruses from the family Adenoviridae. In addition to human hosts, these viruses can be found in, but are not limited to, avian, bovine, porcine and canine species. Certain aspects contemplate the use of any adenovirus from any of the four genera of the family Adenoviridae (e.g., Aviadenovirus, Mastadenovirus, Atadenovirus and Siadenovirus) as the basis of an E2b deleted virus vector, or vector containing other deletions as described herein. In addition, several serotypes are found in each species. Ad also pertains to genetic derivatives of any of these viral serotypes, including but not limited to, genetic mutation, deletion or transposition of homologous or heterologous DNA sequences.

The term “antigen” or “Ag”, and its grammatical equivalents as used herein, can refer to a molecule that provokes the immune response. This immune response can involve either antibody production, or the activation of specific immunologically-competent cells, or both. The skilled artisan will understand that any macromolecule, including virtually all proteins or peptides, can serve as an antigen. The term “immunoglobulin” or “Ig”, as used herein can refer to a class of proteins, which function as antibodies. Antibodies expressed by B cells are sometimes referred to as the chimeric antigen receptor or antigen receptor. The five members included in this class of proteins are IgA, IgG, IgM, IgD, and IgE, of which IgG is the most common circulating antibody. It is the most efficient immunoglobulin in agglutination, complement fixation, and other antibody responses, and is important in defense against bacteria and viruses. For example, a tumor cell antigen can be recognized by a CAR.

The term “autologous” and its grammatical equivalents as used herein can refer to as originating from the same being. For example, a sample (e.g., cells) can be removed, processed, and given back to the same subject (e.g., patient) at a later time. An autologous process is distinguished from an allogenic process where the donor and the recipient are different subjects.

A “helper adenovirus” or “helper virus” refers to an Ad that can supply viral functions that a particular host cell cannot (the host can provide Ad gene products such as E1 proteins). This virus is used to supply, in trans, functions (e.g., proteins) that are lacking in a second virus, or helper dependent virus (e.g., a gutted or gutless virus, or a virus deleted for a particular region such as E2b or other region as described herein); the first replication-incompetent virus is said to “help” the second, helper dependent virus thereby permitting the production of the second viral genome in a cell.

The term “Adenovirus 5 null (Ad5-null),” as used herein, refers to a non-replicating Ad that does not contain any heterologous nucleic acid sequences for expression.

The term “First Generation adenovirus,” as used herein, refers to an Ad that has the early region 1 (E1) deleted. In additional cases, the nonessential early region 3 (E3) can also be deleted.

The term “chimeric antigen receptor” or “CAR” as used herein refers to an engineered molecule, which when expressed by T cells, redirect the T cells to kill a target cell with a specificity dictated by the artificial receptor. Most commonly, the CAR's extracellular binding domain is derived from a murine, humanized, or fully human monoclonal antibody.

The term “epitope” and its grammatical equivalents as used herein can refer to a part of an antigen that can be recognized by antibodies, B cells, T cells or engineered cells. For example, an epitope can be a cancer epitope that is recognized by a TCR. Multiple epitopes within an antigen can also be recognized. The epitope can also be mutated.

The term “engineered” and its grammatical equivalents as used herein can refer to one or more alterations of a nucleic acid, e.g., the nucleic acid within an organism's genome. The term “engineered” can refer to alterations, additions, and/or deletion of genes. An engineered cell can also refer to a cell with an added, deleted and/or altered gene.

The term “cell” or “engineered cell” and their grammatical equivalents as used herein can refer to a cell of human or non-human animal origin. An engineered cell can also refer to the CAR-expressing cell in certain aspects.

The term “good manufacturing practices” (GMP) and its grammatical equivalents as used herein can refer to products that are safe, effective, or pure according to the FDA. GMP can also sometimes be referred to as “cGMP.” The “c” stands for “current.” Manufacturers of a product can employ technologies and systems which are up-to-date in order to comply with regulation of GMP products. GMP compatible products can be utilized in the clinical setting as opposed to the research setting.

The term “gutted” or “gutless,” as used herein, refers to an adenovirus vector that has been deleted of all viral coding regions.

The term “transfection” as used herein refers to the introduction of foreign nucleic acid into eukaryotic cells. Transfection can be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign nucleic acid, DNA or RNA, into the genome of the transfected cell. The term “stable transfectant” refers to a cell which has stably integrated foreign DNA into the genomic DNA.

The term “reporter gene” indicates a nucleotide sequence that encodes a reporter molecule (including an enzyme). A “reporter molecule” is detectable in any of a variety of detection systems, including, but not limited to enzyme-based detection assays (e.g., ELISA, as well as enzyme-based histochemical assays), fluorescent, radioactive, and luminescent systems. In one embodiment, the E. coli β-galactosidase gene (available from Pharmacia Biotech, Pistacataway, N.J.), green fluorescent protein (GFP) (commercially available from Clontech, Palo Alto, Calif.), the human placental alkaline phosphatase gene, the chloramphenicol acetyltransferase (CAT) gene or other reporter genes that are known to the art can be employed.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The nucleic acid sequence thus codes for the amino acid sequence.

The term “heterologous nucleic acid sequence”, as used herein, refers to a nucleotide sequence that is ligated to, or is manipulated to become ligated to, a nucleic acid sequence to which it is not ligated in nature, or to which it is ligated at a different location in nature. Heterologous nucleic acid can include a nucleotide sequence that is naturally found in the cell into which it is introduced or the heterologous nucleic acid can contain some modification relative to the naturally occurring sequence.

The term “transgene” refers to any gene coding region, either natural or heterologous nucleic acid sequences or fused homologous or heterologous nucleic acid sequences, introduced into the cells or genome of a test subject. In the current invention, transgenes are carried on any viral vector that is used to introduce the transgenes to the cells of the subject.

The term “Second Generation Adenovirus”, as used herein, refers to an Ad that has all or parts of the E1, E2, E3, and, in certain embodiments, E4 DNA gene sequences deleted (removed) from the virus.

The term “subject”, as used herein, refers to any animal, e.g., a mammal or marsupial. Subjects include but are not limited to humans, non-human primates (e.g., rhesus or other types of macaques), mice, pigs, horses, donkeys, cows, sheep, rats, and fowl of any kind.

The term “peripheral blood lymphocytes” (PBL) and its grammatical equivalents as used herein can refer to lymphocytes that circulate in the blood (e.g., peripheral blood). Peripheral blood lymphocytes can refer to lymphocytes that are not localized to organs. Peripheral blood lymphocytes can comprise T cells, NK cells, B cell, or any combinations thereof.

The term “recipient” and their grammatical equivalents as used herein can refer to a human or non-human animal. The recipient can also be in need thereof.

The term “T cell” and its grammatical equivalents as used herein can refer to a T cell from any origin. For example, a T cell can be a primary T cell, e.g., an autologous T cell, a cell line, etc. The T cell can also be human or non-human.

The term “T cell activation” or “T cell triggering” and its grammatical equivalents as used herein can refer to the state of a T cell that has been sufficiently stimulated to induce detectable cellular proliferation, cytokine production and/or detectable effector function. In the context of the current invention, “full T cell activation” is similar to triggering T cell cytotoxicity. T cell activation can be measured using various assays known in the art. Assays can be an ELISA to measure cytokine secretion, an ELISPOT, flow cytometry assays to measure intracellular cytokine expression (CD107), flow cytometry assays to measure proliferation, and cytotoxicity assays (51Cr release assay) to determine target cell elimination. Assays typically use controls (non-engineered cells) to compare to engineered cells (CAR T) to determine relative activation of an engineered cell compared to a control. Additionally, assays can compare engineered cells incubated or put in contact with a target cell not expressing the target antigen. For example, comparison can be a CD19-CAR T cell incubated with a target cell that does not express CD19.

The term “sequence” and its grammatical equivalents as used herein can refer to a nucleotide sequence, which can be DNA or RNA; can be linear, circular or branched; and can be either single-stranded or double stranded. A sequence can be mutated. A sequence can be of any length, for example, between 2 and 1,000,000 or more nucleotides in length (or any integer value there between or there above), e.g., between about 100 and about 10,000 nucleotides or between about 200 and about 500 nucleotides.

### Compositions and Methods

Disclosed herein are compositions and methods useful for genetically modifying cells and nucleic acids for therapeutic applications. The compositions and methods described throughout can

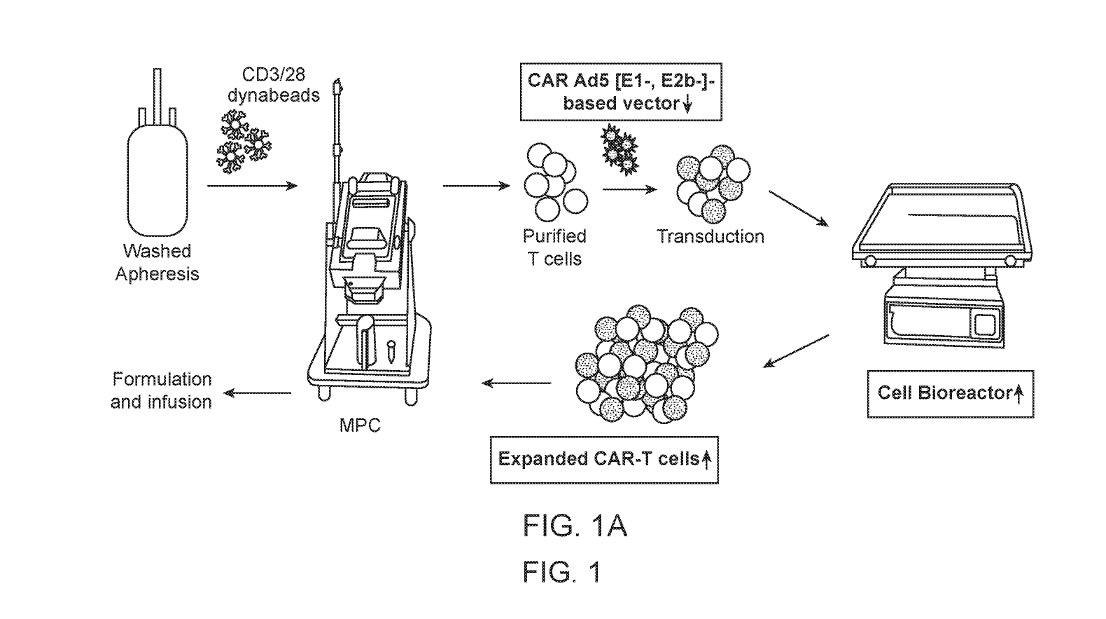

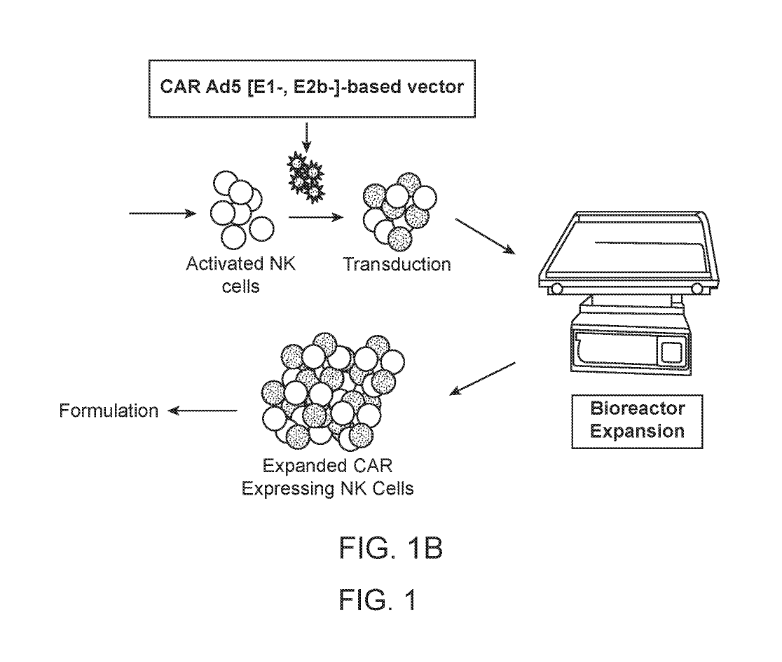

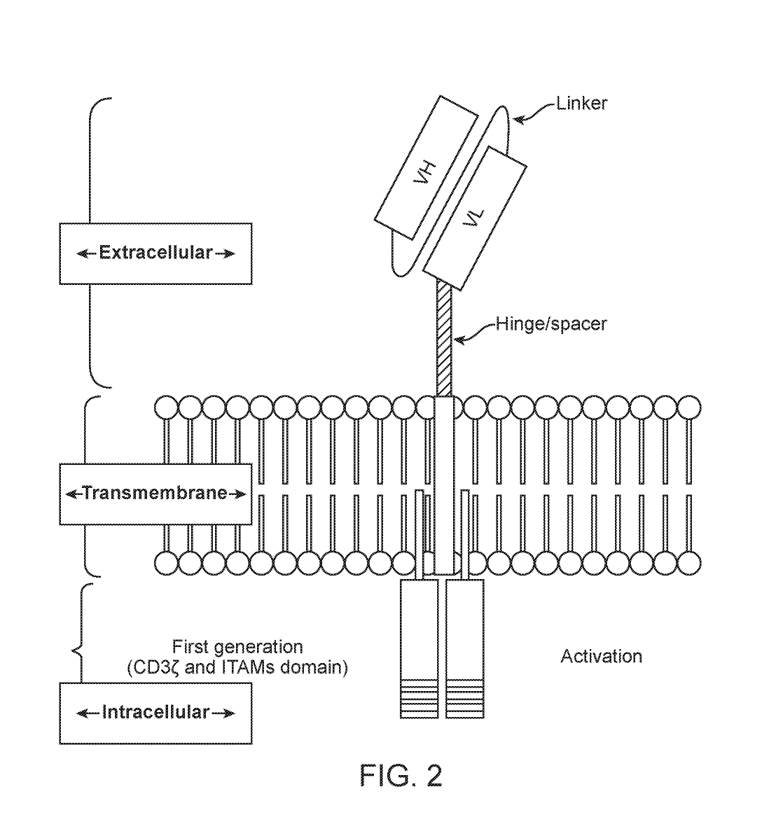

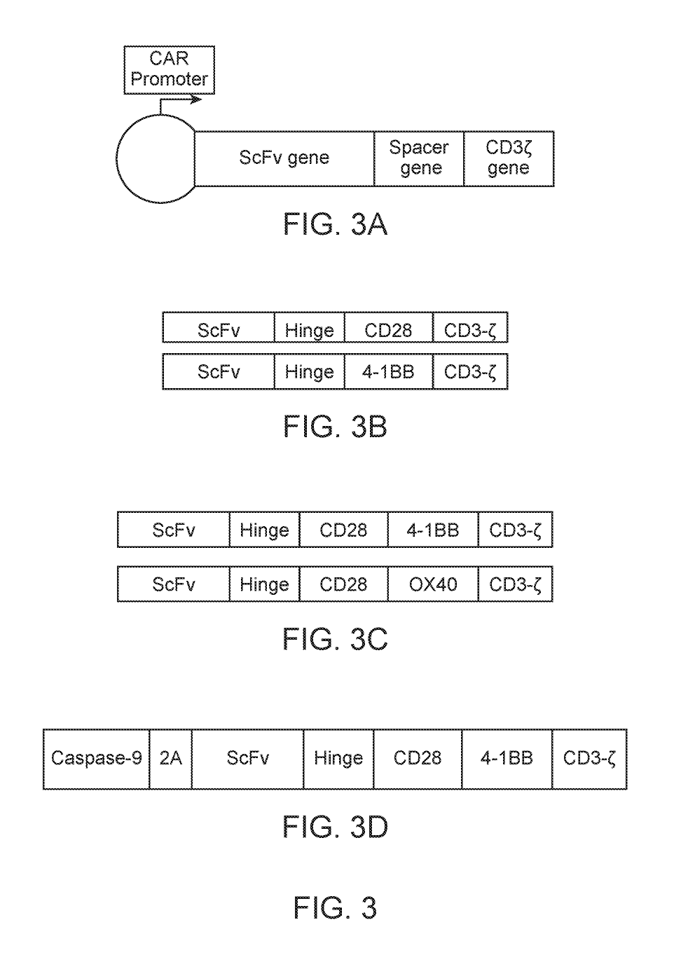

WO 2017/123956 PCT/US2017/013455 use a nucleic acid-mediated genetic engineering process for tumor-specific CAR expression. Effective adoptive cell transfer-based immunotherapies (ACT) can be useful to treat cancer (e.g., metastatic cancer) patients. For example, autologous peripheral blood lymphocytes (PBL) can be modified using non-viral or viral methods to express a chimeric antigen receptor (CAR) that recognizes unique antigens on cancer cells and can be used in the disclosed compositions and methods. Certain aspects are directed to compositions and methods for immunotherapy, including but not limited to cancer, using a human or humanized chimeric antigen receptor (FIG. 1). This chimeric antigen receptor makes use of human or humanized chimeric antigen receptor constructs. The human or humanized chimeric antigen receptor can be combined with a CD8 or CD28 transmembrane portion or functional equivalent thereof, and a signaling region that controls T cell activation fused to a co-stimulatory domain.

A cell can be grown and expanded in a condition that maintains its immunologic and anti-tumor potency and can further be administered into a patient for cancer treatment. A cell can also be grown and expanded in conditions that can improve its performance once administered to a patient. A cell can be selected. For example, prior to expansion and engineering of a cell, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. For example, any T cell lines can be used. Alternatively, a cell can be derived from a healthy donor, from a patient diagnosed with cancer, or from a patient diagnosed with an infection. In another embodiment, a cell can be part of a mixed population of cells which present different phenotypic characteristics. A cell line can also be obtained from a transformed T cell according to the method previously described. A cell can also be obtained from a cell therapy bank. Modified cells resistant to an immunosuppressive treatment can be obtained by any of the method described herein. A desirable cell population can also be selected prior to modification. An engineered cell population can also be selected after modification. An engineered cell can be used in autologous transplantation. Alternatively, a cell can be used in allogeneic transplantation. In some instances, a cell is administered to the same patient whose sample was used to identify the cancer-related target sequence. In other instances, a cell is administered to a patient different from the patient whose sample was used to identify the cancer-related target sequence.

One or more cytokines can be introduced into cells. Cytokines can be utilized to boost transferred cells (including adoptively transferred tumor-specific cells) to expand within a tumor microenvironment. In some cases, IL-2 can be used to facilitate expansion of the cells described herein. Cytokines such as IL-15 can also be employed. Other relevant cytokines in the field of immunotherapy can also be utilized, such as IL-2, IL-7, IL-12, and, L-21, or any combination thereof. In some cases, recombinant cytokines are used.

These compositions and methods can provide a cancer therapy with many advantages. In particular aspects there can be provided a method of modifying T cells to render cells substantially non-dependent upon the presence or activity of the major histocompatibility complex (MHC).

In a further apsect, there can be provided a polynucleic acid described herein encoding for an engineered chimeric antigen receptor.

In still further aspects, there can be provided method of making cells.

In a certain aspect, there can be provided a method to treat a patient in need thereof with the cells described herein.

### Cell Engineering

A nucleic acid, e.g., DNA, encoding a transgene sequence can be randomly inserted into a chromosome of a cell. A random integration can result from any method of introducing a nucleic acid, e.g., DNA, into a cell. For example, the method can be, but is not limited to, electroporation, sonoporation, use of a gene gun, lipotransfection, calcium phosphate transfection, use of dendrimers, microinjection, and use of viral vectors including adenoviral, AAV, and retroviral vectors, and/or group II ribozymes.

A DNA encoding a transgene can also be designed to include a reporter gene so that the presence of a transgene or its expression product can be detected via activation of the reporter gene. Any reporter gene can be used, such as those disclosed above. By selecting in cell culture those cells in which a reporter gene has been activated, cells can be selected that contain a transgene.

A transgene to be inserted can be flanked by engineered sites analogous to a targeted double strand break site in the genome to excise the transgene from a polynucleic acid so it can be inserted at the double strand break region.

A DNA encoding a transgene can be introduced into a cell via electroporation. A DNA can also be introduced into a cell via lipofection, infection, or transformation. Electroporation and/or lipofection can be used to transfect primary cells. Electroporation and/or lipofection can be used to transfect primary hematopoietic cells. A DNA can also be introduced into a cell genome without the use of homologous recombination. In some cases, a DNA can be flanked by engineered sites that are complementary to the targeted double strand break region in a genome. In some cases, a DNA can be excised from a polynucleic acid so it can be inserted at a double strand break region without homologous recombination.

Expression of a CAR can be verified by an expression assay, for example, qPCR or by measuring levels of RNA. Expression level can be indicative also of copy number. For example, if expression levels are extremely high, this can indicate that more than one copy of a CAR was integrated in a genome. Alternatively, high expression can indicate that a transgene was integrated in a highly transcribed area, for example, near a highly expressed promoter. Expression can also be verified by measuring protein levels, such as through Western blotting.

In some cases, the transgene construct comprising the nucleic acid encoding an immunoglobulin alpha chain and beta chain is coexpressed with a separate transgene construct comprising a signaling complex. In some cases, the immunoglobulin transgene and the signaling complex transgene can be present on the same expression vector. In some cases, the immunoglobulin transgene and the signaling complex transgene can be present on the different expression vectors. In the latter case, the two expression vectors can be introduced to a cell either at the same time or sequentially. In some cases, a cell is activated prior to introduction of a vector. In some cases, a cell is activated using anti-CD3 and anti-CD28. In some cases, cytokines are also used to activate a cell.

In some cases, a vector encoding for transgenes comprising a CAR and a signaling complex can be introduced using two distinct methods. In some cases, one vector is introduced virally and another is introduced non-virally. In some cases, one vector is introduced randomly and a second is introduced using a targeted technique. In some cases, a vector can repair a double strand break in the genome.

Transgenes can be useful for expressing, e.g., overexpressing, endogenous genes at higher levels than without a transgene. Additionally, transgenes can be used to express exogenous genes at a level greater than background, i.e., a cell that has not been transfected with a transgene. Transgenes can also encompass other types of genes, for example, a dominant negative gene.

Transgenes can be placed into an organism, cell, tissue, or organ, in a manner which produces a product of a transgene. A polynucleic acid can comprise a transgene.

A T cell can comprise one or more transgenes. One or more transgenes can express a CAR protein recognizing and binding to at least one epitope (e.g., cancer epitope) on an antigen or bind to a mutated epitope on an antigen. A CAR can be a functional CAR. A T cell can also comprise one or more CARs. A T cell can also comprise a single CAR and a secondary engineered receptor.

The present functional CAR protein can be directed against any presented epitope. The present functional CAR fusion protein can also have a peptide-based or peptide-guided function in order to target an antigen. The present functional CAR can be linked, for example, the present functional TCR can be linked with a 2A sequence. The present functional CAR can also be linked with a 2A sequence. The present functional CAR can also contain mammalian components. For example, the present functional CAR can contain mouse constant regions. The present functional CAR can also in some cases contain human constant regions. The peptide-guided function can in principle be achieved by introducing peptide sequences into a CAR and by targeting tumors with these peptide sequences. These peptides can be derived from phage display or synthetic peptide library (see e.g., Arap, W., et al., “Cancer Treatment by Targeted Drug Delivery to Tumor Vasculature in a Mouse Model,” Science, 279, 377-380 (1998); Scott, C. P., et al., “Structural requirements for the biosynthesis of backbone cyclic peptide libraries,” 8: 801-815 (2001)). Among others, peptides specific for breast, prostate and colon carcinomas as well as those specific for neo-vasculatures were already successfully isolated and can be used (Samoylova, T. I., et al., “Peptide Phage Display: Opportunities for Development of Personalized Anti-Cancer Strategies,” Anti-Cancer Agents in Medicinal Chemistry, 6(1): 9-17(9) (2006)). The present functional CAR protein can be directed against a mutated cancer epitope or mutated cancer antigen.

Transgenes that can be used and are specifically contemplated can include those genes that exhibit a certain identity and/or homology to genes disclosed herein, for example, a CAR gene. Therefore, it is contemplated that if a gene exhibits at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology (at the nucleic acid or protein level), it can be used as a transgene. It is also contemplated that a gene that exhibits at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity (at the nucleic acid or protein level) can be used as a transgene. In some cases, the transgene can be functional.

Transgene can be incorporated into a cell. For example, a transgene can be incorporated into an organism's germ line. When inserted into a cell, a transgene can be either a complementary DNA (cDNA) segment, which is a copy of messenger RNA (mRNA), or a gene itself residing in its original region of genomic DNA (with or without introns). A transgene of protein X can refer to a transgene comprising a nucleotide sequence encoding protein X. As used herein, in some cases, a transgene encoding protein X can be a transgene encoding 100% or about 100% of the amino acid sequence of protein X. In other cases, a transgene encoding protein X can be a transgene encoding at least or at least about 99%, 98%, 97%, 96%, 95%, 94%, 93%, 92%, 91%, 90%, 85%, 80%, 75%, 70%, 65%, 60%, 55%, 50%, 40%, 30%, 20%, 10%, 5%, or 1% of the amino acid sequence of protein X. Expression of a transgene can ultimately result in a functional protein, e.g., a partially, fully, or overly functional protein. As discussed above, if a partial sequence is expressed, the ultimate result can be a nonfunctional protein or a dominant negative protein. A nonfunctional protein or dominant negative protein can also compete with a functional (endogenous or exogenous) protein. A transgene can also encode RNA (e.g., mRNA, shRNA, siRNA, or microRNA). In some cases, where a transgene encodes for an mRNA, this can in turn be translated into a polypeptide (e.g., a protein). Therefore, it is contemplated that a transgene can encode for protein. A transgene can, in some instances, encode a protein or a portion of a protein. Additionally, a protein can have one or more mutations (e.g., deletion, insertion, amino acid replacement, or rearrangement) compared to a wild-type polypeptide. A protein can be a natural polypeptide or an artificial polypeptide (e.g., a recombinant polypeptide). A transgene can encode a fusion protein formed by two or more polypeptides. A T cell can comprise or can comprise about 1, 2, 3, 4, 5, 6, 7, 8, 9, 10, 11, 12, 13, 14, 15, 16, 17, 18, 19, 20, or more transgenes. For example, a T cell can comprise one or more transgene comprising a CAR gene.

A T cell can comprise one or more disrupted genes and one or more transgenes. For example, one or more genes whose expression is disrupted can comprise any one of CD27, CD40, CD122, OX40, GITR, CD137, CD28, ICOS, A2AR, B7-H3, B7-H4, BTLA, CTLA-4, IDO, KIR, LAG3, PD-1, TIM-3, VISTA, and/or any combination thereof. For example, solely to illustrate various combinations, one or more genes whose expression is disrupted can comprise PD-1 and one or more transgenes comprise TCR. In another example, one or more genes whose expression is disrupted can also comprise CTLA-4, and one or more transgenes comprise TCR.

A T cell can comprise one or more suppressed genes and one or more transgenes. For example, one or more genes whose expression is suppressed can comprise any one of CD27, CD40, CD122, OX40, GITR, CD137, CD28, ICOS, A2AR, B7-H3, B7-H4, BTLA, CTLA-4, IDO, KIR, LAG3, PD-1, TIM-3, VISTA, and/or any combination thereof. For example, solely to illustrate various combinations, one or more genes whose expression is suppressed can comprise PD-1 and one or more transgenes comprise CAR. In another example, one or more genes whose expression is suppressed can also comprise CTLA-4, and one or more transgenes can comprise any engineered receptor.

A transgene can be a suicide gene. As evidenced in many effective treatments in cancer patients, objective tumor regressions in response to CAR Ts can be accompanied by toxicities. In some cases, a CAR T cell may be unable to distinguish between tumor and normal tissues when the targeted antigen is shared between them (“on-target/off-tumor” toxicity). In other cases, systemic perturbation of the immune system, known as cytokine release syndrome (CRS), can occur. CRS can comrpise systemic inflammatory response syndrome or cytokine storm, which can be consequences of the rapid in vivo expansion of CAR T cells. CRS is a condition characterized by fever and hypotension that, in severe cases, can lead to multiple organ failure. In some cases, toxicity can correlate with in vivo expansion of infused CAR T cells, which can cause a general perturbation of the immune system, and release of high levels of pro-inflammatory cytokines, such as TNF alpha and IL-6.

In some cases, CAR T cells targeting antigens shared with normal tissues can be generated such that they transiently express the CAR, for example after electroporation of mRNA encoding the receptor. In addition, there are significant efforts to further engineer CAR T cells by including safety switches that can allow the drastic elimination of CAR T cells in case of severe on-target toxicity. Vectors in which a CAR is combined with safety switches, such as the inducible caspase-9 gene (activated by a chemical inducer of dimerization) or a truncated form of the EGF receptor R (activated by the monoclonal antibody cetuximab) can be used.

A CAR T cell can encode a suicide gene transgene. A transgene can also comprise a CAR receptor or another similar receptor. A suicide gene can induce elimination of CAR T cells. A suicide gene can be any gene that induces apoptosis in CAR T cells. A suicide gene can be encoded within an adenoviral vector transgene along with the CAR.

One or more transgenes can be from different species. For example, one or more transgenes can comprise a human gene, a mouse gene, a rat gene, a pig gene, a bovine gene, a dog gene, a cat gene, a monkey gene, a chimpanzee gene, or any combination thereof. For example, a transgene can be from a human, having a human genetic sequence. One or more transgenes can comprise human genes. In some cases, one or more transgenes are not adenoviral genes.

A transgene can be inserted into a genome of a T cell in a random or site-specific manner, as described above. For example, a transgene can be inserted to a random locus in a genome of a T cell. These transgenes can be functional, e.g., fully functional if inserted anywhere in a genome. For instance, a transgene can encode its own promoter or can be inserted into a position where it is under the control of an endogenous promoter. Alternatively, a transgene can be inserted into a gene, such as an intron of a gene or an exon of a gene, a promoter, or a non-coding region. A transgene can be inserted such that the insertion disrupts a gene, e.g., an endogenous immune checkpoint.

Sometimes, more than one copy of a transgene can be inserted into more than a random locus in a genome. For example, multiple copies can be inserted into a random locus in a genome. This can lead to increased overall expression than if a transgene was randomly inserted once. Alternatively, a copy of a transgene can be inserted into a gene, and another copy of a transgene can be inserted into a different gene. A transgene can be targeted so that it could be inserted to a specific locus in a genome of a T cell.

Expression of a transgene can be controlled by one or more promoters. A promoter can be a ubiquitous, constitutive (unregulated promoter that allows for continual transcription of an associated gene), tissue-specific promoter or an inducible promoter. Expression of a transgene that is inserted adjacent to or near a promoter can be regulated. For example, a transgene can be inserted near or next to a ubiquitous promoter. Some ubiquitous promoters can be a CAGGS promoter, an hCMV promoter, a PGK promoter, an SV40 promoter, or a ROSA26 promoter.

A promoter can be endogenous or exogenous. For example, one or more transgenes can be inserted adjacent or near to an endogenous or exogenous ROSA26 promoter. Further, a promoter can be specific to a T cell. For example, one or more transgenes can be inserted adjacent or near to a porcine ROSA26 promoter.

Tissue specific promoter or cell-specific promoters can be used to control the location of expression. For example, one or more transgenes can be inserted adjacent or near to a tissue-specific promoter. Tissue-specific promoters can be a FABP promoter, an Lck promoter, a CamKII promoter, a CD19 promoter, a Keratin promoter, an Albumin promoter, an aP2 promoter, an insulin promoter, an MCK promoter, a MyHC promoter, a WAP promoter, or a Co12A promoter.

Tissue specific promoter or cell-specific promoters can be used to control the location of expression. For example, one or more transgenes can be inserted adjacent or near to a tissue-specific promoter. Tissue-specific promoters can be a FABP promoter, an Lck promoter, a CamKII promoter, a CD19 promoter, a Keratin promoter, an Albumin promoter, an aP2 promoter, an insulin promoter, an MCK promoter, a MyHC promoter, a WAP promoter, or a Co12A promoter.

Inducible promoters can be used as well. These inducible promoters can be turned on and off when desired, by adding or removing an inducing agent. It is contemplated that an inducible promoter can be, but is not limited to, a Lac, tac, trc, trp, araBAD, phoA, recA, proU, cst-1, tetA, cadA, nar, PL, cspA, T7, VHB, Mx, and/or Trex.

### Engineered Receptors

Engineered receptors can be used in cells, compositions, or method described herein, including, but not be limited to, a chimeric antigen receptor (CAR), a T-cell receptor (TCR), or a B-cell receptor (BCR), or a derivative thereof.

In certain aspects, the chimeric antigen receptor can be comprised of an extracellular antigen recognition domain, a trans-membrane domain, and a signaling region that controls T cell activation. The extracellular antigen recognition domain can be derived from a murine, a humanized or fully human monoclonal antibody. Specifically, the extracellular antigen recognition domain is comprised of the variable regions of the heavy and light chains of a monoclonal antibody that is cloned in the form of single-chain variable fragments (scFv) and joined through a hinge and a transmembrane domain to an intracellular signaling molecule of the T-cell receptor (TCR) complex and at least one co-stimulatory molecule. In some cases, a co-stimulatory domain is not used.

A CAR can be present in the plasma membrane of a eukaryotic cell, e.g., a mammalian cell, where suitable mammalian cells include, but are not limited to, a cytotoxic cell, a T lymphocyte, a stem cell, a progeny of a stem cell, a progenitor cell, a progeny of a progenitor cell, and an NK cell. When present in the plasma membrane of a eukaryotic cell, a CAR can be active in the presence of its binding target. A target can be expressed on a membrane. A target can also be soluble (e.g., not bound to a cell). A target can be present on the surface of a cell such as a target cell. A target can be presented on a solid surface such as a lipid bilayer; and the like. A target can be soluble, such as a soluble antigen. A target can be an antigen. An antigen can be present on the surface of a cell such as a target cell. An antigen can be presented on a solid surface such as a lipid bilayer; and the like. In some cases, a target can be an epitope of an antigen.

Extracellular binding region. In certain aspects, the chimeric antigen receptor can have an extracellular antigen recognition domain. In one embodiment, the extracellular antigen recognition domain can be fully human. In other cases, the extracellular antigen recognition domain can be humanized. In other cases, the extracellular antigen recognition domain can be murine. In some cases, the extracellular antigen recognition domain can be non-human. The moieties used to bind to antigen can fall into three general categories: single-chain variable fragments (scFv's) derived from antibodies, fragment antigen binding region (Fab) selected from libraries, or nature ligands that engage their cognate receptor. The binding region can encompass a scFv, a Fab, or a nature ligand, as well as any of their derivatives.

The scFv can be the portion of the CAR that determines its antigen specificity. The scFv can bind to any complementary target. The scFv used can be derived from an antibody for which the sequences of the variable regions are known. The scFv used can be derived from an antibody sequence obtained from an available mouse hybridoma. The scFv used can be obtained from whole-exomic sequencing of a tumor cell or primary cell.

By employing genetic engineering, a scFv can be modified in a variety of ways. In some cases, a scFv can be mutated, so that the scFv can be selected for higher affinity to its target. In some cases, the affinity of the scFv for its target can be optimized for targets that can be expressed at low levels on normal tissues. This optimization can be performed to minimize potential toxicities. In other cases, the cloning of a scFv that has a higher affinity for the membrane bound form of a target can be preferable over its soluble form counterpart. This modification can be performed because some targets can also be detected in soluble form at different levels and their targeting can cause unintended toxicity.

Hinge or Spacer. In certain aspects, a CAR used herein can comprise a hinge. A hinge can also be called a spacer. In certain aspects, a hinge can be considered a portion of a CAR used to provide flexibility to a scFv. In some cases, a hinge can be used to detect a CAR on the cell surface of a cell, particularly when antibodies to detect the scFv are not functional or available. For instance the length of the hinge derived from an immunoglobulin can require optimization depending on the location of the epitope on the target that the scFv is targeting.

In some cases, a hinge may not belong to an immunoglobulin but instead to another molecule such the native hinge of a CD8 alpha molecule. A CD8 alpha hinge can contain cysteine and proline residues known to play a role in the interaction of a CD8 co-receptor and MHC molecule. Cystein and proline residues can influence the performance of a CAR.

A CAR hinge can be size tunable and can compensate to some extent in normalizing the orthogonal synapse distance between CAR T cell and a target cell. This topography of the immunological synapse between a T cell and a target cell also defines a distance that cannot be functionally bridged by a CAR due to a membrane-distal epitope on a cell-surface target molecule that, even with a short hinge CAR, cannot bring the synapse distance in to an approximation for signaling. Likewise, membrane-proximal CAR target antigen epitopes have been described for which signaling outputs are only observed in the context of a long hinge CAR. A hinge can be tuned according to the single chain variable fragment region that is used. A hinge can be of any length.

Transmembrane region. In certain aspects, the transmembrane motif can anchor the CAR to the plasma membrane of a cell. A native transmembrane portion of CD28 can be used in a CAR. In other cases, a native transmembrane portion of CD8 alpha can also be used in the CAR.

Intracellular signaling region. The signaling domain of a CAR used herein can be responsible for activation of at least one of the normal effector functions of the immune cell in which the CAR has been placed in. A CAR can induce the effector function of a T cell, for example, can be cytolytic activity or helper activity including the secretion of cytokines. Thus, the term “signaling domain” can refer to the portion of a protein which transduces the effector function signal and directs the cell to perform a specialized function. While usually the entire signaling domain can be employed, in many cases it is not necessary to use the entire chain. In some cases, a truncated portion of the intracellular signaling domain is used. In some cases, the term signaling domain is thus meant to include any truncated portion of the intracellular signaling domain sufficient to transduce the effector function signal. Preferred examples of signaling domains for use in a CAR include a cytoplasmic sequence of the T cell receptor (TCR) and co-receptors that act in concert to initiate signal transduction following target-receptor engagement, as well as any derivative or variant of these sequences and any synthetic sequence that has the same functional capability.

In certain aspects, the design of a CAR can comprise a simple incorporation of the zeta-chain (1^{st} generation CAR) to the incorporation either of a single co-stimulatory domain (for example, CD28 or 4-1BB) (2^{nd} generation), or the incorporation of two co-stimulatory endodomains (CD28/OX40 or CD28/4-1BB) (3^{rd} generation). Together with co-receptors such as CD8, these signaling moieties produce downstream activation of kinase pathways, which support gene transcription and functional cellular responses. Co-stimulatory endodomains of CARs can activate proximal signaling proteins related to either CD28 (Phosphatidylinositol-4,5-bisphosphate 3-kinase) or 4-1BB/OX40 (TNF-receptor-associated-factor adapter proteins) pathways, and MAPK and Akt activation.

In some cases, signaling domains can contain signaling motifs which are known as immunoreceptor tyrosine-based activation motifs (ITAMs). Examples of ITAM containing cytoplasmic signaling sequences include those derived from TCR zeta, FcR gamma, FcR beta, CD3 gamma, CD3 delta, CD3 epsilon, CD5, CD22, CD79a, CD79b, and CD66d. However, in preferred embodiments, the intracellular signaling domain is derived from CD3 zeta chain.

An example of a T cell signaling domain containing one or more ITAM motifs is the CD3 zeta domain, also known as T-cell receptor T3 zeta chain or CD247. This domain is part of the T-cell receptor-CD3 complex and plays an important role in coupling antigen recognition to several intracellular signal-transduction pathways with primary effector activation of the T cell. As used herein, CD3 zeta is primarily directed to human CD3 zeta and its isoforms as known from Swissprot entry P20963, including proteins having a substantially identical sequence. As part of the chimeric antigen receptor, again the full T cell receptor T3 zeta chain is not required and any derivatives thereof comprising the signaling domain of T-cell receptor T3 zeta chain are suitable in the methods in certain aspects, including any functional equivalents thereof.

In some cases, signals generated through the CAR can be complexed with secondary or co-stimulatory signals. With respect to the co-stimulatory signaling domain, the chimeric antigen receptor like complex can be designed to comprise several possible co-stimulatory signaling domains. As is well known in the art, in naïve T-cells the mere engagement of the T-cell receptor is not sufficient to induce full activation of T-cells into cytotoxic T-cells. Full, productive T cell activation requires a second co-stimulatory signal. Several receptors that have been reported to provide co-stimulation for T-cell activation, include, but are not limited to CD28, OX40, CD27, CD2, CD5, ICAM-1, LFA-1 (CD11a/CD18), and 4-1BB. The signaling pathways utilized by these co-stimulatory molecules share the common property of acting in synergy with the primary T cell receptor activation signal. These co-stimulatory signaling regions provide a signal that is synergistic with the primary effector activation signal, originating from one or more ITAM motifs, for example a CD3 zeta signaling domain, and can complete the requirements for activation of the T cell.

In certain aspects, the addition of co-stimulatory domains to the chimeric antigen receptor like complex can enhance the efficacy and durability of the engineered cells.

In another embodiment the T cell signaling domain and the co-stimulatory domain are fused to one another thereby composing the signaling region.

CAR function. In some instances, a CAR, when present on the plasma membrane of a cell, and when activated by binding its target, can result in cytotoxic activity by the cell toward a target that expresses on its cell surface an antigen to which the binding domain of the CAR binds. For example, in some cases a cell can be a cytotoxic cell (e.g., an NK cell or a cytotoxic T lymphocyte), a CAR of the present disclosure, when present in the plasma membrane of a cell, and when activated by binding its target, can increase cytotoxic activity of a cytotoxic cell toward a target cell that expresses on its cell surface an antigen to which the binding domain of a CAR binds. For example, in some cases a cell can be an NK cell or a T lymphocyte, a CAR of the present disclosure, when present in the plasma membrane of a cell, and when activated by binding of its target, can increase cytotoxic activity of a cell by at least 10%, at least 15%, at least 20%, at least 25%, at least 30%, at least 40%, at least 50%, at least 75%, at least 2-fold, at least 2.5-fold, at least 5-fold, at least 10-fold, or more 10-fold, compared to the cytotoxic activity of the cell in the absence of the binding target.

In some cases, a CAR, when activated by binding its target, can result in other CAR activation related events such as proliferation and expansion (either due to increased cellular division or anti-apoptotic responses). In some cases, a CAR, when activated by binding its target, can result in other CAR activation related events such as intracellular signaling modulation, cellular differentiation, or cell death.

A binding region can also comprise a single-chain variable fragment (scFv). The extracellular domain or ecto-domain of an exemplary CAR consists of the scFv from the antigen binding sites of a monoclonal antibody, thereby linking the V_{H} and V_{L} domains. The scFv is linked to a flexible trans-membrane domain followed by one or more endo-domains that can include a tyrosine-based activation motif such as that from CD3 zeta. In the so-called second and third generation CARs, additional activation domains from co-stimulatory molecules such as CD28 and CD137 (4-1BB), which serve to enhance T cell survival and proliferation, were included.

Some recent advances have focused on identifying tumor-specific mutations that in some cases trigger an antitumor T cell response. For example, these endogenous mutations can be identified using a whole-exomic-sequencing approach. Tran E, et al., “Cancer immunotherapy based on mutation-specific CD4+ T cells in a patient with epithelial cancer,” Science 344: 641-644 (2014). Therefore, a CAR can be comprised of a scFv targeting a tumor-specific mutation.

A method can identify a cancer-related target sequence from a sample obtained from a cancer patient using an in vitro assay (e.g., whole-exomic sequencing). A method can further identify a TCR transgene from a first T cell that recognizes the target sequence. A cancer-related target sequence and a TCR transgene can be obtained from samples of the same patient or different patients. A cancer-related target sequence can be encoded on a CAR transgene to render a CAR specific to a target sequence. A method can effectively deliver a nucleic acid comprising a CAR transgene across a membrane of a T cell. In some instances, the first and second T cells can be obtained from the same patient. In other instances, the first and second T cells can be obtained from different patients. In other instances, the first and second T cells can be obtained from different patients. The method can safely and efficiently integrate a CAR transgene into the genome of a T cell using a non-viral integration or a viral integration system to generate an engineered T cell and thus, a CAR transgene can be reliably expressed in the engineered T cell.

### Cancer Targets

In certain aspects, the cell or the engineered receptor can bind a target antigen. An engineered cell can target an antigen. An engineered cell can also target an epitope.

The target antigen can be a tumor cell neo-antigen, a tumor neo-epitope, tumor-specific antigen, a tumor associated antigen, a tissue-specific antigen, a bacterial antigen, a viral antigen, a yeast antigen, a fungal antigen, a protozoan antigen, a parasite antigen, a mitogen, or a combination thereof. An antigen can be a tumor cell antigen. An epitope can be a tumor cell epitope. Such a tumor cell epitope can be derived from a wide variety of tumor antigens such as antigens from tumors resulting from mutations, shared tumor specific antigens, differentiation antigens, and antigens overexpressed in tumors. Those antigens, for example, can be derived from Neo-antigens, Neo-antigen epitopes, folate receptor alpha, WT1, p53, Brachyury, brachyury (TIVS7-2, polymorphism), brachyury (IVS7 T/C polymorphism), T brachyury, T, hTERT, hTRT, iCE, HPV E6, HPV E7, MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MC1R, Gp100, PSA, PSMA, PSCA, STEAP, PAP, Tyrosinase, TRP-1, TRP-2, ART-4, CAMEL, Cyp-B, EGFR, HER1, HER2/neu, HER3, HER4, hTERT, hTRT, iCE, mucin 1 (MUC1), MUC1 (VNTR polymorphism), MUC1-c, MUC1-n, MUC2, PRAME, P15, RU1, RU2, SART-1, SART-3, WT1, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, TEL/AML1, carcinoembryonic antigen (CEA), human epidermal growth factor receptor 2 (HER2/neu), human epidermal growth factor receptor 3 (HER3), Human papillomavirus (HPV), MUC1, Prostate-specific antigen (PSA), alpha-actinin-4, ARTC1, CAR-ABL fusion protein (b3a2), B-RAF, CASP-5, CASP-8, beta-catenin, Cdc27, CDK4, CDKN2A, COA-1, dek-can fusion protein, EFTUD2, Elongation factor 2, ETV6-AML1 fusion protein, FLT3-ITD, FN1, GPNMB, LDLR-fucosyltransferase fusion protein, HLA-A2d, HLA-Al ld, hsp70-2, KIAAO205, MART2, ME1, MUM-1f, MUM-2, MUM-3, neo-PAP, Myosin class I, NFYC, OGT, OS-9, p53, pml-RARalpha fusion protein, PRDXS, PTPRK, K-ras, N-ras, RBAF600, SIRT2, SNRPD1, SYT-SSX1- or -SSX2 fusion protein, TGF-betaRII, triosephosphate isomerase, BAGE-1, GAGE-1, 2, 8, Gage 3, 4, 5, 6, 7, GnTVf, HERV-K-MEL, KK-LC-1, KM-HN-1, LAGE-1, MAGE-A1, MAGE-A2, MAGE-A3, MAGE-A4, MAGE-A6, MAGE-A9, MAGE-A10, MAGE-A12, MAGE-C2, mucink, NA-88, NY-ESO-1/LAGE-2, SAGE, Sp17, SSX-2, SSX-4, TAG-1, TAG-2, TRAG-3, TRP2-INT2g, XAGE-1b, gp100/Pmel17, Kallikrein 4, mammaglobin-A, Melan-A/MART-1, NY-BR-1, OA1, PSA, RAB38/NY-MEL-1, TRP-1/gp75, TRP-2, tyrosinase, adipophilin, AIM-2, ALDH1A1, BCLX (L), BCMA, BING-4, CPSF, cyclin D1, DKK1, ENAH (hMena), EP-CAM, EphA3, EZH2, FGFS, G250/MN/CAIX, HER-2/neu, IL13Ralpha2, intestinal carboxyl esterase, alpha fetoprotein, M-CSFT, MCSP, mdm-2, MMP-2, MUC1, p53, PBF, PRAME, PSMA, RAGE-1, RGSS, RNF43, RU2AS, secernin 1, SOX10, STEAP1, survivin, Telomerase, VEGF, BRCA1, or a modified variant, a splice variant, a functional epitope, an epitope agonist, or any combination thereof, just to name a few. Tumor-associated antigens can be antigens not normally expressed by the host; they can be mutated, truncated, misfolded, or otherwise abnormal manifestations of molecules normally expressed by the host; they can be identical to molecules normally expressed but expressed at abnormally high levels; or they can be expressed in a context or environment that is abnormal. Tumor-associated antigens can be, for example, proteins or protein fragments, complex carbohydrates, gangliosides, haptens, nucleic acids, other biological molecules, or any combinations thereof.

### Additional Targets

An epitope can be a stromal epitope. Such an epitope can be on the stroma of the tumor microenvironment. The antigen can be a stromal antigen. Such an antigen can be on the stroma of the tumor microenvironment. Those antigens and those epitopes, for example, can be present on tumor endothelial cells, tumor vasculature, tumor fibroblasts, tumor pericytes, tumor stroma, and/or tumor mesenchymal cells, just to name a few. Those antigens, for example, can comprise CD34, MCSP, FAP, CD31, PCNA, CD117, CD40, MMP4, and/or Tenascin.

Furthermore, although not required for expression, exogenous sequences can also include transcriptional or translational regulatory sequences, for example, promoters, enhancers, insulators, internal ribosome entry sites, sequences encoding 2A peptides and/or polyadenylation signals.

In some cases, the exogenous sequence (e.g., transgene) comprises a fusion of a protein of interest and, as its fusion partner, an extracellular domain of a membrane protein, causing the fusion protein to be located on the surface of the cell. In some instances, a transgene encodes a CAR wherein a CAR encoding sequence is inserted into a safe harbor such that a TCR is expressed. In some instances, a CAR encoding sequence is inserted into a PD1 and/or a CTLA-4 locus. In other cases, a CAR is delivered to the cell in a lentivirus for random insertion while the PD1- or CTLA-4 specific nucleases can be supplied as mRNAs. In some instances, a CAR is delivered via a viral vector system such as a retrovirus, AAV or adenovirus along with mRNA encoding nucleases specific for a safe harbor (e.g., AAVS1, CCR5, albumin or HPRT). The cells can also be treated with mRNAs encoding PD1 and/or CTLA-4 specific nucleases. In some cases, the polynucleotide encoding a CAR is supplied via a viral delivery system together with mRNA encoding HPRT specific nucleases and PD 1- or CTLA-4 specific nucleases. CARs that can be used with the methods and compositions in certain aspects include all types of these chimeric proteins, including first, second and third generation designs. CARs comprising specificity domains derived from antibodies can be particularly useful, although specificity domains derived from receptors, ligands and engineered polypeptides can be also envisioned. The intercellular signaling domains can be derived from TCR chains such as zeta and other members of the CD3 complex such as the y and E chains. In some cases, CARs can comprise additional co-stimulatory domains such as the intercellular domains from CD28, CD137 (also known as 4-1BB) or CD134. In still further cases, two types of co-stimulator domains can be used simultaneously (e.g., CD3 zeta used with CD28+CD137).

In some cases, an engineered cell can be a stem memory T_{SCM} cell comprised of CD45RO (−), CCR7(+), CD45RA (+), CD62L+ (L-selectin), CD27+, CD28+ and/or IL-7Rα+. Stem memory cells can also express CD95, IL-2Rβ, CXCR3, and/or LFA-1, and show numerous functional attributes distinctive of stem memory cells. Engineered cells can also be central memory T_{CM} cells comprising L-selectin and CCR7, where the central memory cells can secrete, for example, IL-2, but not IFNγ or IL-4. Engineered cells can also be effector memory T_{EM} cells comprising L-selectin or CCR7 and produce, for example, effector cytokines such as IFNγ and IL-4.

Delivery of a Vector into a Cell Membrane

The nucleases and transcription factors, polynucleotides encoding same, and/or any transgene polynucleotides and compositions comprising the proteins and/or polynucleotides described herein can be delivered to a target cell by any suitable means.

Vector Systems. A number of viral based systems have been developed for gene transfer into mammalian cells. For example, retroviruses provide a convenient platform for gene delivery systems. A selected gene can be inserted into a vector and packaged in retroviral particles using techniques known in the art. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells. They also have the added advantage of low immunogenicity. Adenoviral vectors have the advantage that they do not integrate into the genome of the target cell thereby bypassing negative integration-related events.

First generation, or E1-deleted adenovirus vectors Ad5 [E1-] are constructed such that a transgene replaces only the E1 region of genes. For example, about 90% of the wild-type Ad5 genome is retained in the vector. Ad5 [E1-] vectors have a decreased ability to replicate and cannot produce infectious virus after infection of cells not expressing the Ad5 E1 genes. The recombinant Ad5 [E1-] vectors are propagated in human cells (for example, 293 cells but other appropriate cells can also be used) allowing for Ad5 [E1-] vector replication and packaging. Ad5 [E1-] vectors have a number of positive attributes; one of which is their relative ease for scale up and cGMP production. Additionally, Ad5 vectors do not integrate; their genomes can remain episomal. Generally, for vectors that do not integrate into the host genome, the risk for insertional mutagenesis and/or germ-line transmission is extremely low if at all in contract to retroviral and lentiviral based systems. Conventional Ad5 [E1-] vectors have a carrying capacity that approaches 7kb.

The Ad5 [E1-, E2b-] platform has an expanded cloning capacity that can allow inclusion of multiple genes (Hartigan-O'Connor, et al, 2002, Methods Enzymol, 346, 224-246). Ad5 [E1-, E2b-] vectors have up to about 12 kb gene-carrying capacity as compared to the 7 kb capacity of Ad5 └E1-┘ vectors, providing space for multiple genes if needed. In some embodiments, an insert of more than 1, 2, 3, 4, 5, 6, 7, 8, 9, 10, or 11 kb is introduced into an Ad5 vector, such as the Ad5 [E1-, E2b-] vector. Deletion of the E2b region confers advantageous immune properties on the Ad5 vectors, often eliciting potent immune responses to target transgene antigens while minimizing the immune responses to Ad viral proteins.

Certain aspects can contemplate the use of E2b deleted adenovirus vectors utilizing cell lines that express the deleted E2b gene products. Certain aspects can also provide such packaging cell lines; for example, E.C7 (formally called C-7), derived from the HEK-203 cell line, Amalfitano, et al. Proc Natl Acad Sci USA, 1996, 93, 3352-3356; Amalfitano, et al. Gene Ther, 1997, 4,258-263.

The E2b gene products, DNA polymerase and pre-terminal protein, can be constitutively expressed in E.C7, or similar suitable cells along with the E1 gene products. Transfer of gene segments from the Ad genome to a production cell line can have advantages such as increased carrying capacity; and, a decreased potential of replication competent Ad generation, that can require two or more independent recombination events to generate a replication competent Ad. The E1, Ad DNA polymerase and/or pre-terminal protein expressing cell lines used can enable the propagation of adenoviral vectors with a carrying capacity approaching 13 kb, without the need for a contaminating helper virus, Mitani et al, 1995, Proc. Natl. Acad. Sci., 92, 3854; Hodges, et al, 2000, J Gene Med, 2, 250-259.

In certain cases, when genes critical to a viral life cycle are deleted (e.g., the E2b genes), a further crippling of Ad to replicate or express other viral gene proteins occurs. This can decrease immune recognition of virally infected cells, and allow for extended durations of foreign transgene expression.

The transcription factors and nucleases as described herein can be delivered using vectors, for example containing sequences encoding one or more of the proteins. Transgenes encoding polynucleotides can be similarly delivered. Any vector systems can be used including, but not limited to, plasmid vectors, retroviral vectors, lentiviral vectors, adenovirus vectors, poxvirus vectors; herpesvirus vectors and adeno-associated virus vectors, etc. Furthermore, any of these vectors can comprise one or more transcription factor, nuclease, and/or transgene

Conventional viral and non-viral based gene transfer methods can be used to introduce nucleic acids encoding engineered CRISPR/Cas, TALEN, transposon-based, ZEN, meganuclease, or Mega-TAL molecules and/or transgenes in cells (e.g., mammalian cells) and target tissues. Such methods can also be used to administer nucleic acids encoding CRISPR/Cas, TALEN, transposon-based, ZEN, meganuclease, or Mega-TAL molecules and/or transgenes to cells in vitro. In some examples, nucleic acids encoding CRISPR/Cas, TALEN, transposon-based, ZEN, meganuclease, or Mega-TAL molecules and/or transgenes can be administered for in vivo or ex vivo immunotherapy uses. Non-viral vector delivery systems can include DNA plasmids, naked nucleic acid, and nucleic acid complexed with a delivery vehicle such as a liposome or poloxamer. Viral vector delivery systems can include DNA and RNA viruses, which have either episomal or integrated genomes after delivery to the cell.

Methods of non-viral delivery of nucleic acids include electroporation, lipofection, nucleofection, gold nanoparticle delivery, microinjection, biolistics, virosomes, liposomes, immunoliposomes, polycation or lipid: nucleic acid conjugates, naked DNA, mRNA, artificial virions, and agent-enhanced uptake of DNA. Sonoporation using, e.g., the Sonitron 2000 system (Rich-Mar) can also be used for delivery of nucleic acids.

Vectors including viral and non-viral vectors containing nucleic acids encoding engineered CRISPR/Cas, TALEN, transposon-based, ZEN, meganuclease, or Mega-TAL molecules, transposon and/or transgenes can also be administered directly to an organism for transduction of cells in vivo. Alternatively, naked DNA or mRNA can be administered. Administration is by any of the routes normally used for introducing a molecule into ultimate contact with blood or tissue cells including, but not limited to, injection, infusion, topical application and electroporation. More than one route can be used to administer a particular composition. Pharmaceutically acceptable carriers are determined in part by the particular composition being administered, as well as by the particular method used to administer a composition.

Vectors can be used to express a gene, e.g., a transgene, or portion of a gene of interest. A gene of portion or a gene can be inserted by using any method. For example, a method can be a restriction enzyme-based technique.

Vectors can be delivered in vivo by administration to an individual patient, by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, T cells, bone marrow aspirates, tissue biopsy), followed by reimplantation of the cells into a patient, usually after selection for cells which have incorporated the vector. Prior to or after selection, the cells can be expanded.

### Suitable Cells

Prior to expansion and genetic modification of cells, a source of cells can be obtained from a subject. In some cases, T cells can be obtained. T cells can be obtained from a number of sources, including PBMCs, bone marrow, lymph node tissue, cord blood, thymus tissue, and tissue from a site of infection, ascites, pleural effusion, spleen tissue, and tumors.

In certain embodiments, T cells can be obtained from a unit of blood collected from a subject using any number of techniques known to the skilled artisan, such as Ficoll™ separation. In one embodiment, cells from the circulating blood of an individual are obtained by apheresis. The apheresis product can contain lymphocytes, including T cells, monocytes, granulocytes, B cells, other nucleated white blood cells, red blood cells, and platelets. In one embodiment, the cells collected by apheresis can be washed to remove the plasma fraction and to place the cells in an appropriate buffer or media for subsequent processing steps.

The composition of T cell subsets of CAR T products used can be extremely heterogeneous. In a preferred embodiment, cells used can be largely composed of a heterogeneous proportion of CD4 and CD8 T cells. CD4 and CD8 cells can have phenotypic characteristics of circulating effector T cells. CD4 and CD8 cells can also have a phenotypic characteristic of effector-memory cells. In another embodment, cells can be central-memory cells.

Suitable cells can include but are not limited to eukaryotic and prokaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells as well as insect cells such as Spodopterafugiperda (Sf), or fungal cells such as Saccharomyces, Pichia and Schizosaccharomyces. In some cases, the cell line is a CHO-K1, MDCK or HEK293 cell line. In some cases, suitable primary cells include peripheral blood mononuclear cells (PBMC), peripheral blood lymphocytes (PBL), and other blood cell subsets such as, but not limited to, T cell, a natural killer cell, a monocyte, a natural killer T cell, a monocyte-precursor cell, a hematopoietic stem cell or a non-pluripotent stem cell. In some cases, the cell can be any immune cells including any T-cell such as tumor infiltrating cells (TILs), such as CD3+ T-cells, CD4+ T-cells, CD8+ T-cells, or any other type of T-cell. The T cell can also include memory T cells, memory stem T cells, or effector T cells.

The T cells can also be selected from a bulk population, for example, selecting T cells from whole blood. The T cells can also be expanded from a bulk population. The T cells can also be skewed towards particular populations and phenotypes. For example, the T cells can be skewed to phenotypically comprise, CD45RO (−), CCR7 (+), CD45RA (+), CD62L (+), CD27 (+), CD28 (+) and/or IL-7Rα (+).

Suitable cells can be selected that comprise one of more markers selected from a list comprising: CD45RO (−), CCR7 (+), CD45RA (+), CD62L (+), CD27 (+), CD28 (+) and/or IL-7Rα (+).

Suitable cells also include stem cells such as, by way of example, embryonic stem cells, induced pluripotent stem cells, hematopoietic stem cells, neuronal stem cells and mesenchymal stem cells. Suitable cells can comprise any number of primary cells, such as human cells, non-human cells, and/or mouse cells. Suitable cells can be progenitor cells. Suitable cells can be derived from the subject to be treated (e.g., patient). Suitable cells can be derived from a human donor.

Suitable cells can be stem memory T_{SCM} cells comprised of CD45RO (−), CCR7(+), CD45RA (+), CD62L+ (L-selectin), CD27+, CD28+ and IL-7Rα+, stem memory cells can also express CD95, IL-2Rβ, CXCR3, and LFA-1, and show numerous functional attributes distinctive of stem memory cells.

Suitable cells can be central memory T_{CM} cells comprising L-selectin and CCR7, central memory cells can secrete, for example, IL-2, but not IFNγ or IL-4. Suitable cells can also be effector memory T_{EM} cells comprising L-selectin or CCR7 and produce, for example, effector cytokines such as IFNγ and IL-4.

A method of attaining suitable cells can comprise selecting cells. In some cases, a cell can comprise a marker that can be selected for the cell. For example, such marker can comprise GFP, a resistance gene, a cell surface marker, or an endogenous tag. Cells can be selected using any endogenous marker. Suitable cells can be selected using any technology. Such technology can comprise flow cytometry and/or magnetic columns. Selected cells can subsequently be infused into a subject. Selected cells can also be expanded to large numbers. Selected cells can be expanded prior to infusion.

### Pharmaceutical Compositions and Formulations

The compositions described throughout can be formulation into a pharmaceutical medicament and be used to treat a human or mammal, in need thereof, diagnosed with a disease, e.g., cancer. These medicaments can be co-administered with one or more T cells (e.g., engineered T cells) to a human or mammal, together with one or more chemotherapeutic agent or chemotherapeutic compound.

Populations of CAR T cells can be formulated for administration to a subject using techniques known to the skilled artisan. Formulations comprising populations of CAR T cells can include pharmaceutically acceptable excipient(s). Excipients included in the formulations will have different purposes depending, for example, on the subpopulation of T cells used and the mode of administration. Examples of generally used excipients included, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents. The formulations comprising populations of CAR T cells can have been prepared and cultured in the absence of any non-human components, such as animal serum. A formulation can include one population of CAR T cells, or more than one, such as two, three, four, five, six or more populations of CAR T cells.

Formulations comprising population(s) of CAR T cells can be administered to a subject using modes and techniques known to the skilled artisan. Exemplary modes include, but are not limited to, intravenous injection. Other modes include, without limitation, intratumoral, intradermal, subcutaneous (S.C., s.q., sub-Q, Hypo), intramuscular (i.m.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection of infusion of the formulations can be used to effect such administration.

Formulations comprising population(s) of CAR T cells that are administered to a subject comprise a number of CAR T cells that is effective for the treatment and/or prophylaxis of the specific indication or disease. Thus, therapeutically-effective populations of CAR T cells are administered to subjects. In general, formulations are administered that comprise between about 1×10⁴ and about 1×10¹⁰ CAR T cells. In most cases, the formulation will comprise between about 1×10⁵ and about 1×10⁹ CAR T cells, from about 5×10⁵ to about 5×10⁸ CAR T cells, or from about 1×10⁶ to about 1×10⁷ CAR T cells. However, the number of CAR T cells administered to a subject will vary between wide limits, depending upon the location, source, identity, extent and severity of the cancer, the age and condition of the individual to be treated etc. A physician will ultimately determine appropriate dosages to be used.

Tumor-targeting molecules are administered to a subject prior to, or concurrent with, or after administration of the CAR T cells. The tumor-targeting molecules bind to target cells in the subject by association to a tumor-associated antigen or a tumor-specific antigen. The tumor-targeting molecules can be formulated for administration to a subject using techniques known to the skilled artisan. Formulations of the tumor-targeting molecules can include pharmaceutically acceptable excipient(s). Examples of generally used excipients include, without limitation: saline, buffered saline, dextrose, water-for-injection, glycerol, ethanol, and combinations thereof, stabilizing agents, solubilizing agents and surfactants, buffers and preservatives, tonicity agents bulking agents, and lubricating agents.

The tumor-targeting molecules can be administered to a subject using modes and techniques known to the skilled artisan. Exemplary modes include, but are not limited to, intravenous, intraperitoneal, and intratumoral injection. Other modes include, without limitation, intradermal, subcutaneous (S.C., s.q., sub-Q, Hypo), intramuscular (i.m.), intra-arterial, intramedullary, intracardiac, intra-articular (joint), intrasynovial (joint fluid area), intracranial, intraspinal, and intrathecal (spinal fluids). Any known device useful for parenteral injection or infusion of the formulations can be used to effect such administration.

Formulations comprising the tumor-targeting molecules are administered to a subject in an amount that is effective for treating and/or prophylaxis of the specific indication or disease. In general, formulations comprising at least about 0.1 mg/kg to about 100 mg/kg body weight of the tumor-targeting molecules are administered to a subject in need of treatment. In most cases, the dosage is from about 1 mg/kg to about 100 mg/kg body weight of the tagged proteins daily, taking into account the routes of administration, symptoms, etc. A physician will determine appropriate dosages to be used.

In one embodiment, a chimeric antigen receptor is used for stimulating a T cell-mediated immune response. A T cell-mediated immune response is an immune response that involves the activation of T cells. Activated antigen-specific cytotoxic T cells are able to induce apoptosis in target cells displaying epitopes of foreign antigens on their surface, such as for example cancer cells displaying tumor antigens.

In another embodiment, a chimeric antigen receptor is used to provide anti-tumor immunity in the mammal. Due to a T cell-mediated immune response the subject will develop an anti-tumor immunity.

There can be provided methods of treating a subject having cancer comprising administering to a subject in need of treatment one or more formulations of tumor-targeting molecules, wherein these molecules bind to a cancer cell, and administering one or more therapeutically-effective populations of CAR T cells, wherein the CAR T cells bind the tumor-targeting molecules and induce cancer cell death.

Another embodiment relates to methods of treating a subject having cancer comprising administering to a subject in need of treatment one or more therapeutically-effective populations of CAR T cells, wherein the CAR T cells bind to a cancer cell, thereby inducing cancer cell death.

Administration frequencies of both formulations comprising CAR T cells and CAR T cells in combination with tumor-targeting molecules will vary depending on factors that include the disease being treated, the elements comprising the CAR T cells and the tumor-targeting molecules, and the modes of administration. Each formulation can be independently administered 4, 3, 2, or once daily, every other day, every third day, every fourth day, every fifth day, every sixth day, once weekly, every eight days, every nine days, every ten days, bi-weekly, monthly and bi-monthly.

A “chemotherapeutic agent” or “chemotherapeutic compound” and their grammatical equivalents as used herein, can be a chemical compound useful in the treatment of cancer. The chemotherapeutic cancer agents that can be used in combination with the disclosed T cell include, but are not limited to, mitotic inhibitors (vinca alkaloids). These include vincristine, vinblastine, vindesine and Navelbine™ (vinorelbine, 5′-noranhydroblastine). In yet other embodiments, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide. The present disclosure further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that can be used in the methods and compositions disclosed herein includes antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The present disclosure further encompasses other chemotherapeutic cancer agents including without limitation anti-tumor antibodies, dacarbazine, azacytidine, amsacrine, melphalan, ifosfamide and mitoxantrone.

The disclosed cell herein can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed T cell include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; avastin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; CAR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

In some cases, for example, in the compositions, formulations and methods of treating cancer, the unit dosage of the composition or formulation administered can be 5, 10, 15, 20, 25, 30, 35, 40, 45, 50, 55, 60, 65, 70, 75, 80, 85, 90, 95 or 100 mg. In some cases, the total amount of the composition or formulation administered can be 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 40, 50, 60, 70, 80, 90, or 100 g.

In some cases, there can be provided a pharmaceutical composition comprising a T cell can be administered either alone or together with a pharmaceutically acceptable carrier or excipient, by any routes, and such administration can be carried out in both single and multiple dosages. More particularly, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes.

For example, cells can be administered to a patient in conjunction with (e.g., before, simultaneously, or following) any number of relevant treatment modalities, including but not limited to treatment with agents such as antiviral therapy, cidofovir and interleukin-2, or Cytarabine (also known as ARA-C). In some cases, the engineered cells can be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. The engineered cell composition can also be administered to a patient in conjunction with (e.g.,before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH.

In some cases, the engineered cell compositions can be administered following B-cell ablative therapy such as agents that react with CD20, e.g., Rituxan. For example, subjects can undergo standard treatment with high dose chemotherapy followed by peripheral blood stem cell transplantation. In certain embodiments, following the transplant, subjects can receive an infusion of the engineered cells, e.g., expanded engineered cells. Additionally, expanded engineered cells can be administered before or following surgery. The engineered cells obtained by any one of the methods described herein can be used in a particular aspect for treating patients in need thereof against Host versus Graft (HvG) rejection and Graft versus Host Disease (GvHD).

Therefore, there can be provided a method of treating patients in need thereof against Host versus Graft (HvG) rejection and Graft versus Host Disease (GvHD) comprising treating a patient by administering to a patient an effective amount of engineered cells comprising inactivated TCR alpha and/or TCR beta genes.

### Combination Therapy

The compositions comprising CAR T cell immunotherapy compositions as described herein can be formulated into a pharmaceutical medicament and be used to treat a human or mammal in need thereof or diagnosed with a disease, e.g., cancer. These medicaments can be co-administered with one or more additional vaccines or other cancer therapy to a human or mammal.

Natural Killer (NK) Cells

In certain embodiments, native or engineered NK cells may be provided to be administered to a subject in need thereof, in combination with cellular immunotherapy as described herein.

The immune system is a tapestry of diverse families of immune cells each with its own distinct role in protecting from infections and diseases. Among these immune cells are the natural killer, or NK, cells as the body's first line of defense. NK cells have the innate ability to rapidly seek and destroy abnormal cells, such as cancer or virally-infected cells, without prior exposure or activation by other support molecules. In contrast to adaptive immune cells such as T cells, NK cells have been utilized as a cell-based “off-the-shelf” treatment in phase 1 clinical trials, and have demonstrated tumor killing abilities for cancer.

aNK Cells. In addition to native NK cells, there may be provided NK cells for administering to a patient that has do not express Killer Inhibitory Receptors (KIR), which diseased cells often exploit to evade the killing function of NK cells. This unique activated NK, or aNK, cell lack these inhibitory receptors while retaining the broad array of activating receptors which enable the selective targeting and killing of diseased cells. aNK cells also carry a larger pay load of granzyme and perforin containing granules, thereby enabling them to deliver a far greater payload of lethal enzymes to multiple targets.

taNK Cells. Chimeric antigen receptor (CAR) technology is among the most novel cancer therapy approaches currently in development. CARs are proteins that allow immune effector cells to target cancer cells displaying specific surface antigen (target-activated Natural Killer) is a platform in which aNK cells are engineered with one or more CARs to target proteins found on cancers and is then integrated with a wide spectrum of CARs. This strategy has multiple advantages over other CAR approaches using patient or donor sourced effector cells such as autologous T-cells, especially in terms of scalability, quality control and consistency.

Much of the cancer cell killing relies upon ADCC (antibody dependent cell-mediated cytotoxicity) whereupon effector immune cells attach to antibodies, which are in turn bound to the target cancer cell, thereby facilitating killing of the cancer by the effector cell. NK cells are the key effector cell in the body for ADCC and utilize a specialized receptor (CD16) to bind antibodies.

haNK Cells. Studies have shown that perhaps only 20% of the human population uniformly expresses the “high-affinity” variant of CD16, which is strongly correlated with more favorable therapeutic outcomes compared to patients with the “low-affinity” CD16. Additionally, many cancer patients have severely weakened immune systems due to chemotherapy, the disease itself or other factors.

In certain aspects, haNK cells are modified to express high-affinity CD16. As such, haNK cells may potentiate the therapeutic efficacy of a broad spectrum of antibodies directed against cancer cells.

Costimulatory Molecules

Co-stimulatory molecules can be incorporated into the culture during generation of antigen-specific CAR T cells or CAR NK cells, in order to enhance the immunogenicity of the resulting CAR T cell or CAR NK cell immunotherapy compositions. Co-stimulatory domains can also be fused into the chimeric antigen receptor like complex and introduced into the engineered cells. Initiation of an immune response requires at least two signals for the activation of naive T cells by APCs (Damle, et al. J Immunol 148:1985-92 (1992); Guinan, et al. Blood 84:3261-82 (1994); Hellstrom, et al. Cancer Chemother Pharmacol 38:S40-44 (1996); Hodge, et al. Cancer Res 39:5800-07 (1999)). An antigen specific first signal is delivered through the T cell receptor (TCR) via the peptide/major histocompatability complex (MHC) and causes the T cell to enter the cell cycle. A second, or costimulatory, signal may be delivered for cytokine production and proliferation.

At least three distinct molecules normally found on the surface of professional antigen presenting cells (APCs) have been reported as capable of providing the second signal critical for T cell activation: B7-1 (CD80), ICAM-1 (CD54), and LFA-3 (human CD58) (Damle, et al. J Immunol 148:1985-92 (1992); Guinan, et al. Blood 84: 3261-82 (1994); Wingren, et al. Crit Rev Immunol 15: 235-53 (1995); Parra, et al. Scand. J Immunol 38: 508-14 (1993); Hellstrom, et al. Ann NY Acad Sci 690: 225-30 (1993); Parra, et al. J Immunol 158: 637-42 (1997); Sperling, et al. J Immunol 157: 3909-17 (1996); Dubey, et al. J Immunol 155: 45-57 (1995); Cavallo, et al. Eur J Immunol 25: 1154-62 (1995)).

These costimulatory molecules have distinct T cell ligands. B7-1 interacts with the CD28 and CTLA-4 molecules, ICAM-1 interacts with the CD11a/CD18 (LFA-1β2 integrin) complex, and LFA-3 interacts with the CD2 (LFA-2) molecules. Therefore, in a preferred embodiment, it would be desirable to have a recombinant adenovirus vector that contains B7-1, ICAM-1, and LFA-3, respectively, that, when fused with a chimeric antigen receptor to target antigens or antigenic epitopes, enhances the immunogenicity of the resulting engineered cells. TABLE 2 shown in EXAMPLE 3 provides non-limiting examples of co-stimulatory domains, which may be fused with chimeric antigen receptors to generate the engineered cells of this disclosure.

Immune Pathway Checkpoint Modulators

In certain embodiments, immune pathway checkpoint inhibitors are combined with compositions comprising CAR T cell immunotherapies as disclosed herein. In certain embodiments, a patient received an immune pathway checkpoint inhibitor in conjunction with a vaccine or pharmaceutical compositions described herein. In further embodiments, compositions are administered with one or more immune pathway checkpoint modulators. A balance between activation and inhibitory signals regulates the interaction between T lymphocytes and disease cells, wherein T-cell responses are initiated through antigen recognition by the T-cell receptor (TCR). The inhibitory pathways and signals are referred to as immune pathway checkpoints. In normal circumstances, immune pathway checkpoints play a critical role in control and prevention of autoimmunity and also protect from tissue damage in response to pathogenic infection.

Certain embodiments provide combination immunotherapies comprising CAR T cell immunotherapy compositions for modulating immune pathway checkpoint inhibitory pathways for the prevention and/or treatment of cancer and infectious diseases. In some embodiments, modulating can be increasing expression or activity of a gene or protein. In some embodiments, modulating can be decreasing expression or activity of a gene or protein. In some embodiments, modulating can affect a family of genes or proteins.

In general, the immune inhibitory pathways are initiated by ligand-receptor interactions. It is now clear that in diseases, the disease can co-opt immune-checkpoint pathways as mechanism for inducing immune resistance in a subject.

The induction of immune resistance or immune inhibitory pathways in a subject by a given disease can be blocked by molecular compositions such as siRNAs, antisense, small molecules, mimic, a recombinant form of ligand, receptor or protein, or antibodies (which can be an Ig fusion protein) that are known to modulate one or more of the Immune Inhibitory Pathways. For example, preliminary clinical findings with blockers of immune-checkpoint proteins, such as Cytotoxic T-lymphocyte-associated antigen 4 (CTLA4) and programmed cell death protein 1 (PD1) have shown promise for enhancing anti-tumor immunity.

Because diseased cells can express multiple inhibitory ligands, and disease-infiltrating lymphocytes express multiple inhibitory receptors, dual or triple blockade of immune pathway checkpoints proteins may enhance anti-disease immunity. Combination immunotherapies as provide herein can comprise one or more compositions comprising an immune pathway checkpoint modulator that targets one or more of the following immune-checkpoint proteins: PD1, PDL1, PDL2, CD28, CD80, CD86, CTLA4, B7RP1, ICOS, B7RPI, B7-H3 (also known as CD276), B7-H4 (also known as B7-S1, B7x and VCTN1), BTLA (also known as CD272), HVEM, KIR, TCR, LAG3 (also known as CD223), CD137, CD137L, OX40, OX4OL, CD27, CD70, CD40, CD40L, TIM3 (also known as HAVcr2), GALS, A2aR and Adenosine.

In some embodiments, the molecular composition comprises siRNAs. In some embodiments, the molecular composition comprises a small molecule. In some embodiments, the molecular composition comprises a recombinant form of a ligand. In some embodiments, the molecular composition comprises a recombinant form of a receptor. In some embodiments, the molecular composition comprises an antibody. In some embodiments, the combination therapy comprises more than one molecular composition and/or more than one type of molecular composition. As it will be appreciated by those in the art, future discovered proteins of the immune checkpoint inhibitory pathways are also envisioned to be encompassed by the present disclosure.

In some embodiments, combination immunotherapies comprise molecular compositions for the modulation of CTLA4. In some embodiments, combination immunotherapies comprise molecular compositions for the modulation of PD1. In some embodiments, combination immunotherapies comprise molecular compositions for the modulation of PDL1. In some embodiments, combination immunotherapies comprise molecular compositions for the modulation of LAG3. In some embodiments, combination immunotherapies comprise molecular compositions for the modulation of B7-H3. In some embodiments, combination immunotherapies comprise molecular compositions for the modulation of B7-H4. In some embodiments, combination immunotherapies comprise molecular compositions for the modulation of TIM3. In some embodiments, modulation is an increase or enhancement of expression. In other embodiments, modulation is the decrease of absence of expression.

Two non-limiting exemplary immune pathway checkpoint inhibitors include the cytotoxic T lymphocyte associated antigen-4 (CTLA-4) and the programmed cell death protein-1 (PD1). CTLA-4 can be expressed exclusively on T-cells where it regulates early stages of T-cell activation. CTLA-4 interacts with the co-stimulatory T-cell receptor CD28 which can result in signaling that inhibits T-cell activity. Once TCR antigen recognition occurs, CD28 signaling may enhances TCR signaling, in some cases leading to activated T-cells and CTLA-4 inhibits the signaling activity of CD28. The present disclosure provides immunotherapies as provided herein in combination with anti-CTLA-4 monoclonal antibody for the prevention and/or treatment of cancer and infectious diseases. The present disclosure provides vaccine or immunotherapies as provided herein in combination with CTLA-4 molecular compositions for the prevention and/or treatment of cancer and infectious diseases.

Programmed death cell protein ligand-1 (PDL1) is a member of the B7 family and is distributed in various tissues and cell types. PDL1 can interact with PD1 inhibiting T-cell activation and CTL mediated lysis. Significant expression of PDL1 has been demonstrated on various human tumors and PDL1 expression is one of the key mechanisms in which tumors evade host anti-tumor immune responses. Programmed death-ligand 1 (PDL1) and programmed cell death protein-1 (PD1) interact as immune pathway checkpoints. This interaction can be a major tolerance mechanism which results in the blunting of anti-tumor immune responses and subsequent tumor progression. PD1 is present on activated T cells and PDL1, the primary ligand of PD1, is often expressed on tumor cells and antigen-presenting cells (APC) as well as other cells, including B cells. PDL1 interacts with PD1 on T cells inhibiting T cell activation and cytotoxic T lymphocyte (CTL) mediated lysis. The present disclosure provides immunotherapies as provided herein in combination with anti-PD1 or anti-PDL1 monoclonal antibody for the prevention and/or treatment of cancer and infectious diseases.

Certain embodiments may provide immunotherapies as provided herein in combination with PD1 or anti-PDL1 molecular compositions for the prevention and/or treatment of cancer and infectious diseases. Certain embodiments may provide immunotherapies as provided herein in combination with anti-CTLA-4 and anti-PD1 monoclonal antibodies for the prevention and/or treatment of cancer and infectious diseases. Certain embodiments may provide immunotherapies as provided herein in combination with anti-CTLA-4 and PDL1 monoclonal antibodies. Certain embodiments may provide vaccine or immunotherapies as provided herein in combination with anti-CTLA-4, anti-PD1, anti-PDL1 monoclonal antibodies, or a combination thereof, for the treatment of cancer and infectious diseases.

Immune pathway checkpoint molecules can be expressed by T cells. Immune pathway checkpoint molecules can effectively serve as “brakes” to down-modulate or inhibit an immune response. Immune pathway checkpoint molecules include, but are not limited to Programmed Death 1 (PD1 or PD-1, also known as PDCD1 or CD279, accession number: NM_005018), Cytotoxic T-Lymphocyte Antigen 4 (CTLA-4, also known as CD152, GenBank accession number AF414120.1), LAG3 (also known as CD223, accession number: NM_002286.5), Tim3 (also known as hepatitis A virus cellular receptor 2 (HAVCR2), GenBank accession number: JX049979.1), B and T lymphocyte associated (BTLA) (also known as CD272, accession number: NM_181780.3), BY55 (also known as CD160, GenBank accession number: CR541888.1), TIGIT (also known as IVSTM3, accession number: NM_173799), LAIR1 (also known as CD305, GenBank accession number: CR542051.1), SIGLECIO (GenBank accession number: AY358337.1), natural killer cell receptor 2B4 (also known as CD244, accession number: NM_001166664.1), PPP2CA, PPP2CB, PTPN6, PTPN22, CD96, CRTAM, SIGLEC7, SIGLEC9, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, ILIORA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3 which directly inhibit immune cells. For example, PD1 can be combined with a CAR T cell immunotherapy composition to treat a patient in need thereof.

Additional immune pathway checkpoints that can be targeted can be adenosine A2a receptor (ADORA), CD276, V-set domain containing T cell activation inhibitor 1 (VTCN1), indoleamine 2,3-dioxygenase 1 (IDO1), killer cell immunoglobulin-like receptor, three domains, long cytoplasmic tail, 1 (KIR3DL1), V-domain immunoglobulin suppressor of T-cell activation (VISTA), cytokine inducible SH2-containing protein (CISH), hypoxanthine phosphoribosyltransferase 1 (HPRT), adeno-associated virus integration site 1 (AAVS1), or chemokine (C—C motif) receptor 5 (gene/pseudogene) (CCRS), or any combination thereof.

TABLE 1, without being exhaustive, shows exemplary immune pathway checkpoint genes that can be inactivated to improve the efficiency of CAR T cell immunotherapy compositions as described herein. Immune pathway checkpoints gene can be selected from such genes listed in TABLE 1 and others involved in co-inhibitory receptor function, cell death, cytokine signaling, arginine tryptophan starvation, TCR signaling, Induced T-reg repression, transcription factors controlling exhaustion or anergy, and hypoxia mediated tolerance.

TABLE 1
Examples of Immune Pathway Checkpoint Genes

| Gene | NCBI # | | | Genome |
|---|---|---|---|---|
| Symbol | (GRCh38.p2) | Start | Stop | location |
| ADORA2A | 135 | 24423597 | 24442360 | 22q11.23 |
| CD276 | 80381 | 73684281 | 73714518 | 15q23-q24 |
| VTCN1 | 79679 | 117143587 | 117270368 | 1p13.1 |
| BTLA | 151888 | 112463966 | 112499702 | 3q13.2 |
| CTLA4 | 1493 | 203867788 | 203873960 | 2q33 |
| IDO1 | 3620 | 39913809 | 39928790 | 8p12-p11 |
| KIR3DL1 | 3811 | 54816438 | 54830778 | 19q13.4 |
| LAG3 | 3902 | 6772483 | 6778455 | 12p13.32 |
| PDCD1 | 5133 | 241849881 | 241858908 | 2q37.3 |
| HAVCR2 | 84868 | 157085832 | 157109237 | 5q33.3 |
| VISTA | 64115 | 71747556 | 71773580 | 10q22.1 |
| CD244 | 51744 | 160830158 | 160862902 | 1q23.3 |
| CISH | 1154 | 50606454 | 50611831 | 3p21.3 |

The combination of an adenoviral-based composition and an immune pathway checkpoint modulator may result in reduction in infection, progression, or symptoms of a disease in treated patients, as compared to either agent alone. In another embodiment, the combination of an adenoviral-based composition and an immune pathway checkpoint modulator may result in improved overall survival of treated patients, as compared to either agent alone. In some cases, the combination of an adenoviral-based composition and an immune pathway checkpoint modulator may increase the frequency or intensity of disease-specific T cell responses in treated patients as compared to either agent alone.

Certain embodiments may also provide the use of immune pathway checkpoint inhibition to improve performance of a CAR T cell immunotherapy composition. Certain immune pathway checkpoint inhibitors may be administered at the time of a CAR T cell immunotherapy composition. Certain immune pathway checkpoint inhibitors may also be administered after the administration of a CAR T cell immunotherapy composition. Immune pathway checkpoint inhibition may occur simultaneously to an adenoviral vaccine administration. Immune pathway checkpoint inhibition may occur 1, 2, 3, 4, 5, 6, 7, 8, 9, 10, 15, 20, 30, 40, 50, or 60 minutes after vaccination. Immune pathway checkpoint inhibition may also occur 1, 2, 3, 4, 5, 6, 7, 8, 9, 10, 11, 12, 13, 14, 15, 16, 17, 18, 19, 20, 21, 22, 23, or 24 hours after the administration of a CAR T cell immunotherapy composition. In some cases, immune inhibition may occur 1, 2, 3, 4, 5, 6, or 7 days after vaccination. Immune pathway checkpoint inhibition may occur at any time before or after the administration of a CAR T cell immunotherapy composition.

In another aspect, there is provided methods involving a vaccine comprising one or more nucleic acids encoding an antigen and an immune pathway checkpoint modulator. For example, there is provided a method for treating a subject having a condition that would benefit from downregulation of an immune pathway checkpoint protein, PD1 or PDL1 for example, and its natural binding partner(s) on cells of the subject.

An immune pathway checkpoint modulator may be combined with a CAR T cell immunotherapy composition comprising one or more nucleic acids encoding any antigen. For example, an antigen can be a tumor antigen, such as antigens or epitopes of HER1, HER2/neu, HERS, HER4, or any combination thereof, or any antigen described herein.

An immune pathway checkpoint modulator may produce a synergistic effect when combined with a CAR T cell immunotherapy composition, such as a vaccine. An immune pathway checkpoint modulator may also produce a beneficial effect when combined with a CAR T cell immunotherapy composition.

### Methods of Use

Cells can be extracted from a human as described herein. Cells can be genetically altered ex vivo and used accordingly. These cells can be used for cell-based therapies. These cells can be used to treat disease in a recipient (e.g., a human). For example, these cells can be used to treat cancer.

Described herein is a method of treating a disease (e.g., cancer) in a recipient comprising transplanting to the recipient one or more cells (including organs and/or tissues) comprising engineered cells. The method disclosed herein can be used for treating or preventing disease including, but not limited to, cancer, cardiovascular diseases, lung diseases, liver diseases, skin diseases, or neurological diseases.

In one embodiment, there can be provided methods for administering a genetically modified T cell expressing a chimeric antigen receptor like complex for the treatment of a patient having cancer or at risk of having cancer using lymphocyte infusion. Preferably, autologous lymphocyte infusion is used in the treatment. Autologous peripheral blood monocytes (PBMCs) are collected from a patient in need of treatment and T cells are activated and expanded using the methods described herein and known in the art and then infused back into the patient. Populations of CAR T cells can be formulated for administration; and wherein the administration to a subject using techniques known to the skilled artisan. Alternatively, allogeneic lymphocyte infusion can be used.

In some cases, populations of engineered cells can be formulated for administration to a subject using techniques known to the skilled artisan. Formulations comprising populations of engineered cells include pharmaceutically acceptable excipient(s). Excipients included in the formulations will have different purposes depending, for example, on the subpopulation of T cells used and the mode of administration. Examples of generally used excipients included, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents. The formulations comprising populations of the engineered cells can have been prepared and cultured in the absence of any non-human components, such as animal serum.

A formulation can include one population of engineered cells or more than one, such as two, three, four, five, six or more. The formulations comprising population(s) of engineered cells can be administered to a subject using modes and techniques known to the skilled artisan. Exemplary modes include, but are not limited to, intravenous injection. Other modes include, without limitation, intratumoral, intradermal, subcutaneous (S.C., s.q., sub-Q, Hypo), intramuscular (i.m.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection of infusion of the formulations can be used to effect such administration.

The formulations comprising population(s) of engineered cells that are administered to a subject can comprise a number of engineered cells that is effective for the treatment and/or prophylaxis of the specific indication or disease. Thus, therapeutically-effective populations of engineered cells can be administered to subjects.

In general, formulations can be administered that comprise between about 1×10⁴ and about 1×10¹⁰ engineered cells. In most cases, the formulation will comprise between about 1×10⁵ and about 1×10⁹ engineered cells, from about 5×10⁵ to about 5×10⁸ engineered cells, or from about 1×10⁶ to about 1×10⁷ engineered cells. However, the number of engineered cells administered to a subject can vary between wide limits, depending upon the location, source, identity, extent and severity of the cancer, the age and condition of the individual to be treated etc. A physician can ultimately determine appropriate dosages to be used.

Tumor-targeting molecules can be administered to a subject prior to, or concurrent with, or after administration of engineered cells. A tumor-targeting molecule can bind to target cells in the subject by association to a tumor-associated antigen or a tumor-specific antigen. Tumor-targeting molecules can be formulated for administration to a subject using techniques known to the skilled artisan. Formulations of the tumor-targeting molecules can include pharmaceutically acceptable excipient(s). Examples of generally used excipients include, without limitation: saline, buffered saline, dextrose, water-for-injection, glycerol, ethanol, and combinations thereof, stabilizing agents, solubilizing agents and surfactants, buffers and preservatives, tonicity agents bulking agents, and lubricating agents. A tumor-targeting molecule can be administered to a subject using modes and techniques known to the skilled artisan. Exemplary modes include, but are not limited to, intravenous, intraperitoneal, and intratumoral injection. Other modes include, without limitation, intradermal, subcutaneous (S.C., s.q., sub-Q, Hypo), intramuscular (i.m.), intra-arterial, intramedullary, intracardiac, intra-articular (joint), intrasynovial (joint fluid area), intracranial, intraspinal, and intrathecal (spinal fluids). Any known device useful for parenteral injection or infusion of the formulations can be used to effect such administration.

Formulations comprising the tumor-targeting molecules are administered to a subject in an amount that is effective for treating and/or prophylaxis of the specific indication or disease. In general, formulations comprising at least about 0.1 mg/kg to about 100 mg/kg body weight of the tumor-targeting molecules are administered to a subject in need of treatment. In most cases, the dosage is from about 1 mg/kg to about 100 mg/kg body weight of the tagged proteins daily, taking into account the routes of administration, symptoms, etc. A physician can determine appropriate dosages to be used.

Transplanting can be by any type of transplanting. Sites can include, but not limited to, liver subcapsular space, splenic subcapsular space, renal subcapsular space, omentum, gastric or intestinal submucosa, vascular segment of small intestine, venous sac, testis, brain, spleen, or cornea. For example, transplanting can be subcapsular transplanting. Transplanting can also be intramuscular transplanting. Transplanting can be intraportal transplanting.

Transplanting can be of one or more cells from a human. For example, the one or more cells can be from an organ, which can be a brain, heart, lungs, eye, stomach, pancreas, kidneys, liver, intestines, uterus, bladder, skin, hair, nails, ears, glands, nose, mouth, lips, spleen, gums, teeth, tongue, salivary glands, tonsils, pharynx, esophagus, large intestine, small intestine, rectum, anus, thyroid gland, thymus gland, bones, cartilage, tendons, ligaments, suprarenal capsule, skeletal muscles, smooth muscles, blood vessels, blood, spinal cord, trachea, ureters, urethra, hypothalamus, pituitary, pylorus, adrenal glands, ovaries, oviducts, uterus, vagina, mammary glands, testes, seminal vesicles, penis, lymph, lymph nodes or lymph vessels. The one or more cells can also be from a brain, heart, liver, skin, intestine, lung, kidney, eye, small bowel, or pancreas. The one or more cells can be from a pancreas, kidney, eye, liver, small bowel, lung, or heart. The one or more cells can be from a pancreas. The one or more cells can be pancreatic islet cells, for example, pancreatic β cells. The one or more cells can be any blood cells, such as peripheral blood mononuclear cell (PBMC), lymphocytes, monocytes or macrophages. The one or more cells can be any immune cells such as lymphocytes, B cells, or T cells.

The method disclosed herein can also comprise transplanting one or more cells, where the one or more cells can be can be any types of cells. For example, the one or more cells can be epithelial cells, fibroblast cells, neural cells, keratinocytes, hematopoietic cells, melanocytes, chondrocytes, lymphocytes (B and T), macrophages, monocytes, mononuclear cells, cardiac muscle cells, other muscle cells, granulosa cells, cumulus cells, epidermal cells, endothelial cells, pancreatic islet cells, blood cells, blood precursor cells, bone cells, bone precursor cells, neuronal stem cells, primordial stem cells, hepatocytes, keratinocytes, umbilical vein endothelial cells, aortic endothelial cells, microvascular endothelial cells, fibroblasts, liver stellate cells, aortic smooth muscle cells, cardiac myocytes, neurons, Kupffer cells, smooth muscle cells, Schwann cells, and epithelial cells, erythrocytes, platelets, neutrophils, lymphocytes, monocytes, eosinophils, basophils, adipocytes, chondrocytes, pancreatic islet cells, thyroid cells, parathyroid cells, parotid cells, tumor cells, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, peritubular cells, sertoli cells, lutein cells, cervical cells, endometrial cells, mammary cells, follicle cells, mucous cells, ciliated cells, non-keratinized epithelial cells, keratinized epithelial cells, lung cells, goblet cells, columnar epithelial cells, dopaminergic cells, squamous epithelial cells, osteocytes, osteoblasts, osteoclasts, dopaminergic cells, embryonic stem cells, fibroblasts and fetal fibroblasts. Further, the one or more cells can be pancreatic islet cells and/or cell clusters or the like, including, but not limited to pancreatic α cells, pancreatic β cells, pancreatic δ cells, pancreatic F cells (e.g., PP cells), or pancreatic ε cells. In one instance, the one or more cells can be pancreatic α cells. In another instance, the one or more cells can be pancreatic β cells.

Donor can be at any stage of development including, but not limited to, fetal, neonatal, young and adult. For example, donor T cells can be isolated from adult human. Donor human T cells can be under the age of 10, 9, 8, 7, 6, 5, 4, 3, 2, or 1 year(s). For example, T cells can be isolated from a human under the age of 6 years. T cells can also be isolated from a human under the age of 3 years. A donor can be older than 10 years.

The method disclosed herein can comprise transplanting. Transplanting can be autotransplanting, allotransplanting, xenotransplanting, or any other transplanting. For example, transplanting can be xenotransplanting. Transplanting can also be allotransplanting.

“Xenotransplantation” and its grammatical equivalents as used herein can encompass any procedure that involves transplantation, implantation, or infusion of cells, tissues, or organs into a recipient, where the recipient and donor are different species. Transplantation of the cells, organs, and/or tissues described herein can be used for xenotransplantation in into humans. Xenotransplantation includes but is not limited to vascularized xenotransplant, partially vascularized xenotransplant, unvascularized xenotransplant, xenodressings, xenobandages, and xenostructures.

“Allotransplantation” and its grammatical equivalents (e.g., allogenic transplantation) as used herein can encompass any procedure that involves transplantation, implantation, or infusion of cells, tissues, or organs into a recipient, where the recipient and donor are the same species but different individuals. Transplantation of the cells, organs, and/or tissues described herein can be used for allotransplantation into humans. Allotransplantation includes but is not limited to vascularized allotransplant, partially vascularized allotransplant, unvascularized allotransplant, allodressings, allobandages, and allostructures.

“Autotransplantation” and its grammatical equivalents (e.g., autologous transplantation) as used herein can encompass any procedure that involves transplantation, implantation, or infusion of cells, tissues, or organs into a recipient, where the recipient and donor is the same individual. Transplantation of the cells, organs, and/or tissues described herein can be used for autotransplantation into humans. Autotransplantation includes but is not limited to vascularized autotransplantation, partially vascularized autotransplantation, unvascularized autotransplantation, autodressings, autobandages, and auto structures.

After treatment (e.g., any of the treatment as disclosed herein), transplant rejection can be improved as compared to when one or more wild-type cells is transplanted into a recipient. For example, transplant rejection can be hyperacute rejection. Transplant rejection can also be acute rejection. Other types of rejection can include chronic rejection. Transplant rejection can also be cell-mediated rejection or T cell-mediated rejection. Transplant rejection can also be natural killer cell-mediated rejection.

“Improving” and its grammatical equivalents as used herein can mean any improvement recognized by one of skill in the art. For example, improving transplantation can mean lessening hyperacute rejection, which can encompass a decrease, lessening, or diminishing of an undesirable effect or symptom.

After transplanting, the transplanted cells can be functional in the recipient. Functionality can in some cases determine whether transplantation was successful. For example, the transplanted cells can be functional for at least or at least about 1, 2, 3, 4, 5, 6, 7, 8, 9, 10 or more days. This can indicate that transplantation was successful. This can also indicate that there is no rejection of the transplanted cells, tissues, and/or organs.

In certain instances, transplanted cells can be functional for at least 1 day. Transplanted cells can also functional for at least 7 day. Transplanted cells can be functional for at least 14 day. Transplanted cells can be functional for at least 21 day. Transplanted cells can be functional for at least 28 day. Transplanted cells can be functional for at least 60 days.

Another indication of successful transplantation can be the days a recipient does not require immunosuppressive therapy. For example, after treatment (e.g., transplantation) provided herein, a recipient can require no immunosuppressive therapy for at least or at least about 1, 2, 3, 4, 5, 6, 7, 8, 9, 10 or more days. This can indicate that transplantation was successful. This can also indicate that there is no rejection of the transplanted cells, tissues, and/or organs.

In some cases, a recipient can require no immunosuppressive therapy for at least 1 day. A recipient can also require no immunosuppressive therapy for at least 7 days. A recipient can require no immunosuppressive therapy for at least 14 days. A recipient can require no immunosuppressive therapy for at least 21 days. A recipient can require no immunosuppressive therapy for at least 28 days. A recipient can require no immunosuppressive therapy for at least 60 days. Furthermore, a recipient can require no immunosuppressive therapy for at least 1, 2, 3, 4, 5, 6, 7, 8, 9, 10, or more years.

Another indication of successful transplantation can be the days a recipient requires reduced immunosuppressive therapy. For example, after the treatment provided herein, a recipient can require reduced immunosuppressive therapy for at least 1, 2, 3, 4, 5, 6, 7, 8, 9, 10 or more days. This can indicate that transplantation was successful. This can also indicate that there is no or minimal rejection of the transplanted cells, tissues, and/or organs.

For example, a recipient can require reduced immunosuppressive therapy for at least 1 day. A recipient can also require reduced immunosuppressive therapy for at least 7 days. A recipient can require reduced immunosuppressive therapy for at least 14 days. A recipient can require reduced immunosuppressive therapy for at least 21 days. A recipient can require reduced immunosuppressive therapy for at least 28 days. A recipient can require reduced immunosuppressive therapy for at least 60 days. Furthermore, a recipient can require reduced immunosuppressive therapy for at least 1, 2, 3, 4, 5, 6, 7, 8, 9, 10 or more years.

“Reduced” and its grammatical equivalents as used herein can refer to less immunosuppressive therapy compared to a required immunosuppressive therapy when one or more wild-type cells is transplanted into a recipient.

Immunosuppressive therapy can comprise any treatment that suppresses the immune system. Immunosuppressive therapy can help to alleviate, minimize, or eliminate transplant rejection in a recipient. For example, immunosuppressive therapy can comprise immuno-suppressive drugs. Immunosuppressive drugs that can be used before, during and/or after transplant, but are not limited to, MMF (mycophenolate mofetil (Cellcept)), ATG (anti-thymocyte globulin), anti-CD154 (CD4OL), anti-CD40 (2C10, ASKP1240, CCFZ533X2201), alemtuzumab (Campath), anti-CD20 (rituximab), anti-IL-6R antibody (tocilizumab, Actemra), anti-IL-6 antibody (sarilumab, olokizumab), CTLA4-Ig (Abatacept/Orencia), belatacept (LEA29Y), sirolimus (Rapimune), everolimus, tacrolimus (Prograf), daclizumab (Ze-napax), basiliximab (Simulect), infliximab (Remicade), cyclosporin, deoxyspergualin, soluble complement receptor 1, cobra venom factor, compstatin, anti C5 antibody (eculizumab/Soliris), methylprednisolone, FTY720, everolimus, leflunomide, anti-IL-2R-Ab, rapamycin, anti-CXCR3 antibody, anti-ICOS antibody, anti-OX40 antibody, and anti-CD122 antibody. Furthermore, one or more than one immunosuppressive agents/drugs can be used together or sequentially. One or more than one immunosuppressive agents/drugs can be used for induction therapy or for maintenance therapy. The same or different drugs can be used during induction and maintenance stages. In some cases, daclizumab (Zenapax) can be used for induction therapy and tacrolimus (Prograf) and sirolimus (Rapimune) can be used for maintenance therapy. Daclizumab (Zenapax) can also be used for induction therapy and low dose tacrolimus (Prograf) and low dose sirolimus (Rapimune) can be used for maintenance therapy. Immunosuppression can also be achieved using non-drug regimens including, but not limited to, whole body irradiation, thymic irradiation, and full and/or partial splenectomy. These techniques can also be used in combination with one or more immuno-suppressive drugs.

Ex vivo cell transfection can also be used for diagnostics, research, or for gene therapy (e.g., via re-infusion of the transfected cells into the host organism). In some cases, cells are isolated from the subject organism, transfected with a nucleic acid (e.g., gene or cDNA), and re-infused back into the subject organism (e.g., patient).

Cells (e.g., engineered cells or engineered primary T cells) before, after, and/or during transplantation can be functional. For example, transplanted cells can be functional for at least or at least about 1, 2, 3, 4, 5, 6, 7, 8, 9, 10, 11, 12, 13, 14, 15, 16, 17, 18, 19, 20, 21, 22, 23, 24, 25, 6, 27, 28, 29, 30, 40, 50, 60, 70, 80, 90, or 100 days after transplantation. Transplanted cells can be functional for at least or at least about 1, 2, 3, 4, 5, 6, 7, 8, 9, 10, 11, or 12 months after transplantation. Transplanted cells can be functional for at least or at least about 1, 2, 3, 4, 5, 6, 7, 8, 9, 10, 15, 20, 25, or 30 years after transplantation. In some cases, transplanted cells can be functional for up to the lifetime of a recipient.

Further, transplanted cells can function at 100% of its normal intended operation. Transplanted cells can also function 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of its normal intended operation.

Transplanted cells can also function over 100% of its normal intended operation. For example, transplanted cells can function 110, 120, 130, 140, 150, 160, 170, 180, 190, 200, 250, 300, 400, 500, 600, 700, 800, 900, 1000 or more % of its normal intended operation.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Generation and Functional Characterization of CEA-Specific CAR T Cells

This example describes the generation of a CEA-specific CAR and its functional expression in human T cells. In particular, a CEA-specific CAR carrying a CD28/CD3ζ signaling domain will be designed and will be functionally evaluated.

Vector Design. Ad5 [E1-, E2b-] was constructed and produced as previously described. The CEA hybridoma will be generated from a BALB/c mouse immunized with cultured human colon cancer cells. The scFv CEA will be isolated from the hybridoma and then cloned in frame with the human IgG1-CH2CH3 domains, the CD28 costimulatory endodomain and the CD3ζ chain into the Ad5 [E1-, E2b-] backbone (Ad5 [E1-, E2b-]-CEA.CAR).

Viral supernatant production. For adenovirus production, suitable packaging cells (E.C7 cells) were plated into 10-cm dishes at 1.2×10⁶ cells per dish. After 24 h, cells were transfected with 10 μg adenoviral vector DNA using a transfection reagent (e.g., FuGENE 9 Promega or X-treme gene 9 Roche Diagnostics) and incubated at 37.0° C. for 72 hours. The conditioned media (viral supernatant) from these cells will then be purified via ultracentrifugation (Millipore) and high-titer viral supernatant isolated.

Expression of the Ad5 [E1-, E2b-]-CEA.CAR in T cells. In order to generate CEA-specific T cells, primary human T cells will be isolated and activated from human peripheral blood mononuclear cells (PBMCs). In particular, human T cell expander beads (Life Technologies) will be used to select CD3⁺ cells from PBMC material and activate 1.5×10⁶ CD3⁺ cells per well in a 24-well plate with 100 IU ml⁻¹ rh-IL-2. After 48 h, 0.2×10⁶ to 0.5×10⁶ activated CD3⁺ cells will be resuspended in 0.5 ml harvested retroviral supernatant and 0.5 ml medium supplemented with rh-IL-2 (100 IU ml⁻¹ final) and transferred to Retronectin (Takara)-coated plates. Plates were centrifuged for 90 minutes at 430 g. 48 hours after transduction, cells will be collected and replated in a 24-well plate with 100 IU ml⁻¹ rh-IL-2 at 0.5×10⁶ /ml.

Detection of the Ad5 [E1-, E2b-]-CEA.CAR in T cells. Conjugated CD3, CD4, CD8, CD45RO, CD62L and CCR7 mAbs (BDBiosciences) will be used to identify T lymphocytes expressing the Ad5 [E1-, E2b-]-CEA.CAR. CAR expression in T lymphocytes was assessed using an antibody recognizing the human IgG1-CH2CH3 fragment (Jackson ImmunoResearch). Analyses will be performed on a FACsCaliber flow cytometer using the BDFACs CellQuestPro software (BD Biosciences).

Cells and Cell Lines. For all assays described below, effector cells, target cells and negative control cells are used. Effector cells generally refer to the T cells transduced with the Ad5 [E1-, E2b-]-CEA.CAR. Target cells are generally tumor cells naturally expressing the target (CEA antigen) to which the CAR is directed. Negative control cells are generally cells that do not express the target (CEA negative) to which the CAR is directed.

### Example 2

### Functional Characterization of T Cells Expressing the Ad5 [E1-, E2b-]-CEA.CAR Intracellular IFN-γ Staining

This example describes the functional characterization of T cells expressing the Ad5 [E1-, E2b-]-CEA.CAR by intracellular IFN-γ staining. To evaluate the potential cytotoxic effect of the transduced T cells, different cytotoxicity assays will be performed. The capacity of the Ad5 [E1-, E2b-]-CEA.CAR-expressing T cells to recognize human colon carcinoma cells and their subsequent activation was tested. In general, activation of T cells expressing the CAR can be measured by IFN-gamma (or comparable cytokines) production after stimulation with the cognate antigen (e.g. CEA). 1×10⁵ CAR Transduced T cells will be incubated with 1×10⁵ CEA+ tumor cells expressing the cognate antigen of the CAR (in this example CEA+ tumor cells together with an anti-CEA specific CAR). After 16 h incubation in the presence of 1 μl ml⁻¹ Golgiplug (BD Biosciences) at 37° C., cells were washed and stained with antibodies against CD3, CD8 (both BD Biosciences) and a suitable life/dead dye (IR dye, Life Technologies). Intracellular levels of IFN-gamma were subsequently determined on single-cell basis by flow cytometry using the Cytofix/Cytoperm kit (BD Biosciences) and an antibody against IFN-gamma (BD Biosciences), according to the manufacturer's guidelines. The data were normalized by correction of percentage of IFN-gamma⁺CD8⁺ T cells with the frequency of Ad5 [E1-, E2b-]-CEA.CAR CD8⁺ T cells as measured by antibodies recognizing the human IgG1-CH2CH3 fragment (Jackson ImmunoResearch).

ELISA. Further, IFN-γ levels in the culture supernatants of T cells transduced with Ad5 [E1-, E2b-]-CEA.CAR and stimulated with CEA+ cells and CEA− (control cells) were measured using ELISA.

⁵¹Chromium-Release Assay Cytotoxicity Assay. To evaluate the potential cytotoxic effect of the transduced T cells, different cytotoxicity assays will be performed. In the ⁵¹Chromium-release assay for cell-mediated cytotoxicity, target cells will be labeled overnight with 100 μCu ⁵¹Cr and incubated for 4-5 h with the transduced T cells in 5 different effector-to-target-ratios (E:T), varying between 30:1 and 0.3:1. Percentage of specific lysis will be calculated as follows: (experimental cpm−basal cpm)/(maximal cpm−basal cpm)×100 with maximal lysis determined in the presence of 5% triton and basal lysis in the absence of effector cells.

CMTMR Cytotoxicity Assay. In another cytotoxicity assay, CEA negative control cells are suspended in medium at a concentration 1.5×10⁶ cells/mL, and the fluorescent dye 5-(and-6)-(((4-chloromethyl) benzoyl) amino) tetramethylrhodamine (CMTMR) (Invitrogen) is added at a concentration of 5 μM. The cells are mixed and then incubated at 37° C. for 30 minutes. The cells were then washed and suspended in cytotoxicity medium. Next, the CEA negative control cells are incubated at 37° C. for 60 minutes. The cells are then washed twice and suspended in cytotoxicity medium. Target cells (CEA+) are suspended in PBS+0.1% BSA at 1×10⁶ cells/mL. The fluorescent dye carboxyfluorescein diacetate succinimidyl ester (CFSE) (Invitrogen) is added to this cell suspension at a concentration of 1 μM. The cells are incubated 10 minutes at 37° C. After the incubation, the labeling reaction was stopped by adding a volume of FBS that is equal to the volume of cell suspension and the cells are incubated for 2 minutes at room temperature. The cells are washed and suspended in cytotoxicity medium.

Subsequently, effector T cells (Ad5 [E1-, E2b-]-CEA.CAR) are washed and suspended at 5×10̂6 cells/mL in cytotoxicity medium. In all experiments, the cytotoxicity of Ad5 [E1-, E2b-]-CEA.CAR T cells is compared to the cytotoxicity of negative control effector T cells from the same patient that was transduced with the negative control CAR T (Ad5 [E1-, E2b-]-CD19.CAR) or cells that are not transduced. For effector T cells and negative control effector T cells, cultures are set up in sterile 5 mL test tubes (BD Biosciences) in duplicate at the following T cell: target cell ratios: 10:1, 3:1, and 1:1. The target cells will be 50,000 from a CEA+ patient. Each culture also contains 50,000 negative control cells. In addition, tubes are set up that contain only target cells plus negative control cells. The cultures are incubated for 4 hours at 37° C. Immediately after the incubation, 7AAD (7-aminoactinomycin D) (BD Pharmingen) is added as recommended by the manufacturer, and flow cytometry acquisition will be performed with a BD FacsCanto II (BD Biosciences). Analysis was performed with FlowJo (Treestar, Inc. Ashland, Oreg.). Analysis is gated on 7AAD-negative (live) cells, and the percentages of live target cells and live negative control cells are determined for each T cell+target cell culture. For each T cell+target cell culture, the percent survival of target cells is determined by dividing the percent live target cells by the percent live negative control cells.

The corrected percent survival of target cells calculated by dividing the percent survival of target cells in each T cell+target cell culture by the ratio of the percent target cells: percent negative control cells in tubes containing only target cells and negative control cells without any effector T cells. This correction is necessary to account for variation in the starting cell numbers and for spontaneous target cell death. Cytotoxicity will be calculated as the percent cytotoxicity of target cells=100-corrected percent survival of target cells. For all effector: target ratios, the cytotoxicity will be determined in duplicate and the results will be averaged.

Proliferation assay. Proliferation of Ad5 [E1-, E2b-]-CEA.CAR T cells after exposure to target cells (CEA+) is determined by carboxyfluorescein succinimidyl ester dilution assays (Hudecek M, Lupo-Stanghellini M T, Kosasih P L, Sommermeyer D, Jensen M C, Rader C et al. CEA.CAR⁺ T lymphocytes will be labeled with 1.5 μmol/L carboxyfluorescein diacetate succinimidyl ester (CFSE; Invitrogen) and plated with irradiated tumor targets (CEA positive and CEA negative lines) at an effector-to-target (E:T) ratio of 5:1. CFSE dilution will be measured on CD4⁺ and CD8⁺ T cells by flow cytometry on day 4 of co-culture.

### Example 3

### Clinical Expansion of CEA-Specific CAR T Cells

This example describes the clinical expansion of CEA-specific CAR T cells. In order to generate a large number of transduced T cells, the cells will be induced to proliferate using a rapid expansion protocol (REP). Prior to being used in REPs, T cells will be started in culture with anti-CD3, anti-CD28 and IL-2 and transduced on the second day after the initiation of culture as detailed above. The cells will be cultured in a 75 cm² flask at 37° C. and 5% CO₂. The cells will be counted and suspended at a concentration of 0.5×10⁶ cells/mL in fresh T cell medium with 300 IU/mL of IL-2 every two days for the remainder of the time they will be kept in culture.

A co-stimulatory domain, including any molecule shown in TABLE 2, is included in the CEA vector described above to enhance the immunogenicity of the resulting CEA-specific CAR T cells.

TABLE 2
Co-Stimulatory Domains

| | Gene | NCBI number | | | |
|---|---|---|---|---|---|
| SEQ ID NO | Symbol | (GRCh38.p2) | Start | Stop | Location in genome |
| 1 | CD27 | 939 | 6444885 | 6451718 | 12p13 |
| 2 | CD28 | 940 | 203706475 | 203738912 | 2q33 |
| 3 | TNFRSF9 | 3604 | 7915871 | 7943165 | 1p36 |
| 4 | TNFRSF4 | 7293 | 1211326 | 1214638 | 1p36 |
| 5 | TNFRSF8 | 943 | 12063330 | 12144207 | 1p36 |
| 6 | CD40LG | 959 | 136648177 | 136660390 | Xq26 |
| 7 | ICOS | 29851 | 203936731 | 203961579 | 2q33 |
| 8 | ITGB2 | 3689 | 44885949 | 44928873 | 21q22.3 |
| 9 | CD2 | 914 | 116754435 | 116769229 | 1p13.1 |
| 10 | CD7 | 924 | 82314865 | 82317604 | 17q25.2-q25.3 |
| 11 | KLRC2 | 3822 | 10430599 | 10435993 | 12p13 |
| 12 | TNFRSF18 | 8784 | 1203508 | 1206709 | 1p36.3 |
| 13 | TNFRSF14 | 8764 | 2556365 | 2565622 | 1p36.32 |
| 14 | HAVCR1 | 26762 | 156979480 | 157069527 | 5q33.2 |
| 15 | LGALS9 | 3965 | 27631148 | 27649560 | 17q11.2 |
| 16 | CD83 | 9308 | 14117256 | 14136918 | 6p23 |

What is claimed is:

1. A cell comprising:
(a) at least one engineered receptor; and
(b) at least one extra-chromosomal adenoviral genome;
wherein the adenoviral genome has at least one deletion in a region of an adenoviral gene and encodes the engineered receptor.

2. The cell of claim 1, wherein the engineered receptor is a chimeric antigen receptor (CAR), a T-cell receptor (TCR), or a B-cell receptor (BCR), or a derivative thereof.

3. The cell of claims 1 and 2, wherein the engineered receptor is a chimeric antigen receptor (CAR).

4. The cell of claims 2 and 3, wherein the CAR is a first generation CAR.

5. The cell of claims 2 and 3, wherein the CAR is a second generation CAR.

6. The cell of claims 2 and 3, wherein the CAR is a third generation CAR.

7. The cell of any one of claims 2 to 6, wherein the CAR comprises an extracellular portion, a transmembrane portion, and an intracellular portion.

8. The cell of claim 7, wherein the intracellular portion comprises at least one T cell co-stimulatory domain.

9. The cell of claim 8, wherein the T cell co-stimulatory domain is selected from the group consisting of CD27, CD28, TNFRS9 (4-1BB), TNFRSF4 (OX40), TNFRSF8 (CD30), CD40LG (CD40L), ICOS, ITGB2 (LFA-1), CD2, CD7, KLRC2 (NKG2C), TNFRS18 (GITR), TNFRSF14 (HVEM), or any combination thereof.

10. The cell of any one of claims 1 to 9, wherein the engineered receptor binds a target.

11. The cell of claim 10, wherein the binding is MHC independent.

12. The cell of claim 10, wherein the binding is MHC dependent.

13. The cell of claims 10 to 12, wherein the binding is specific to a disease-associated target.

14. The cell of claim 13 wherein the disease is cancer.

15. The cell of claim 14, wherein the cancer is a solid tumor.

16. The cell of claim 14, wherein the cancer is a liquid tumor.

17. The cell of any one of claims 1 to 16, wherein the receptor binds a target antigen.

18. The cell of claim 17, wherein the target antigen is a tumor cell neo-antigen, a tumor neo-epitope, tumor-specific antigen, a tumor associated antigen, a tissue-specific antigen, a bacterial antigen, a viral antigen, a yeast antigen, a fungal antigen, a protozoan antigen, a parasite antigen, a mitogen, or a combination thereof.

19. The cell of any one of claims 17-18, wherein the target antigen is selected from the group consisting of carcinoembryonic antigen (CEA), human epidermal growth factor receptor 1 (HER1) human epidermal growth factor receptor 2 (HER2/neu), human epidermal growth factor receptor 3 (HER3), human epidermal growth factor receptor 4 (HER4), human papillomavirus (HPV), mucin 1 (MUC1), prostate-specific antigen (PSA), PSMA, Brachyury, folate receptor alpha, WT1, p53, MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MC1R, Gp100, Tyrosinase, TRP-1, TRP-2, ART-4, CAMEL, Cyp-B, BRCA1, BRACHYURY (TIVS7-2, polymorphism), BRACHYURY (IVS7 T/C polymorphism), T BRACHYURY, T, hTERT, hTRT, iCE, MUC1 (VNTR polymorphism), MUC1c, MUC1n, MUC2, PRAME, P15, RU1, RU2, SART-1, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, or TEL/AML1, or a modified variant, a splice variant, a functional epitope, an epitope agonist,or a combination thereof.

20. The cell of any one of claims 1 to 19, wherein the receptor binds a tumor-associated cell.

21. The cell of claim 20, wherein the tumor-associated cell is selected from the group consisting of a fibroblast, a cancer-stem cell, a pericyte, and a stromal cell.

22. The cell of any one of claims 1 to 21, wherein the cell further comprises a secondary receptor.

23. The cell of claim 22, wherein the secondary receptor is a coxsackie adenoviral receptor.

24. The cell of any one of claims 1 to 23, wherein the extra-chromosomal adenoviral genome is adenovirus serotype 5 (Ad5).

25. The cell of any one of claims 1 to 24, wherein the deletion in a region of an adenoviral gene is a deletion in a region of an early region 1 (E1) gene, a deletion in an early region 2b (E2b) gene, a deletion in an early region 3 (E3) gene, or a combination thereof.

26. The cell of any one of claims 1 to 25, wherein the deletion in a region of an adenovirus gene is a deletion in a region of an early region 2b (E2b) gene.

27. The cell of any one of claims 1 to 26, wherein the deletion in a region of an adenoviral gene is a deletion in an early region 1 (E1) gene, an early region 2b (E2b) gene, and early region 3 (E3) gene.

28. The cell of any one of claims 1 to 27, wherein the cell further comprises an exogenous gene.

29. The cell of claim 28, wherein the exogenous gene is selected from a list comprising a suicide gene, a cytokine gene, an anti-angiogenic gene, a metabolism gene, or a hypoxia gene.

30. The cell of any one of claims 1 to 29, wherein the cell further comprises an endogenous gene deletion.

31. The cell of any one of claims 1 to 30, wherein the cell is an immune cell.

32. The cell of claim 31, wherein the immune cell is a T cell.

33. The cell of claim 32, wherein the T cell is an effector (T_{EFF}) cell, effector-memory (T_{EM}) cell, central-memory (T_{CM}), T memory stem (T_{SCM}), naive (T_{N}), or CD4+ or CD8+.

34. The cell of any one of claims 1 to 33, wherein the cell is a primate cell.

35. The cell of any one of claims 1 to 34, wherein the cell is a human cell.

36. The cell of any one of claims 1 to 35, wherein the cell is expanded ex vivo.

37. The cell of any one of claims 1 to 36, wherein the cell is formulated into a pharmaceutical composition.

38. The cell of any one of claims 1 to 37, wherein the cell is part of a combination therapy to treat a subject in need thereof.

39. The cell of any one of claims 1 to 38, wherein the engineered receptor is integrated into the genome of the subject in need thereof.

40. A method of preparing a cell, comprising contacting a cell ex vivo with at least one engineered extrachromosomal vector comprising at least one exogenous receptor sequence.

41. The method of claim 40, wherein the extrachromosomal vector is an adenoviral vector.

42. The method of claim 41, wherein the adenoviral vector is adenovirus serotype 5 (Ad5).

43. The method of any one of claims 40-42, wherein the vector has at least one gene deletion.

44. The method of claim 43, wherein the deletion is a deletion in a region of an early region 1 (E1) gene, and an early region 3 (E3) gene.

45. The method of claim 43, wherein the deletion is a deletion in an early region 2b (E2b) gene, a deletion in an early region 3 (E3) gene, or a combination thereof.

46. The method of any one of claims 40 to 45, wherein the vector contains deletions in an early region 1 (E1) gene, an early region 2b (E2b) gene, and early region3 (E3) gene.

47. The method of any one of claims 40 to 46, wherein the vector is not a gutted vector.

48. The method of any one of claims 40 to 47, wherein the method further comprises introducing at least one secondary receptor before the exogenous receptor.

49. The method of claim 48, wherein the secondary receptor is coxsackie adenovirus receptor.

50. The method of any one of claims 40 to 49, wherein the exogenous receptor sequence is selected from a list comprising a chimeric antigen receptor (CAR), a T-cell receptor (TCR), or a B-cell receptor (BCR), or a derivative thereof.

51. The method of claim 50, wherein the exogenous receptor sequence encodes a chimeric antigen receptor (CAR).

52. The method of any one of claims 40 to 51, wherein the vector further comprises a second exogenous gene sequence.

53. The method of claim 52, wherein the exogenous gene sequence is selected the group consisting of a suicide gene, a cytokine gene, an anti-angiogenic gene, a metabolism gene, and a hypoxia gene.

54. The method of any one of claims 40 to 53, wherein the second exogenous gene sequence comprises an inducible suicide gene sequence.

55. The method of claim 54, wherein the inducible suicide gene sequence is an inducible caspase 9 gene sequence or a portion of the EGF receptor R sequence.

56. The method of any one of claims 40 to 55, wherein the exogenous receptor sequence is introduced into the cell with at least one vector.

57. The method of any one of claims 40 to 56, wherein the cell is activated ex vivo.

58. The method of claim 57, wherein the activation occurs before the exogenous receptor sequence is introduced.

59. The method of any one of claims 57 to 58, wherein the activation is performed with anti-CD3 (OKT3), anti-CD28, at least one cytokine, or any combination thereof.

60. The method of claim 59, wherein the cytokine comprises interleukin-2 (IL-2), interleukin-7 (IL-7), interleukin-15 (IL-15), interleukin-21 (IL-21), or any combination thereof.

61. The method of claims 40 to 60, further comprising expanding the cell.

62. The method of any one of claims 40 to 61, wherein the cell is autologous to a subject in need thereof.

63. The method of any one of claims 40 to 61, wherein the cell is allogenic to a subject in need thereof.

64. The method of any one of claims 62 to 63, wherein the subject in need thereof has preexisting immunity to the adenoviral vector.

65. The method of any one of claims 40 to 64, wherein the cell is a good manufacturing practices (GMP) compatible reagent.

66. The method of claim 65, wherein the reagent is part of a combination therapy to treat cancer.

67. A pharmaceutical composition comprising the cell of any one of claims 1 to 39 or a cell prepared according to any one of claims 40-66.

68. A method of treating a condition in a subject in need thereof comprising administering to a subject a therapeutically effective amount of the pharmaceutical composition of claim 67.

69. The method of claim 68, wherein the pharmaceutical composition is administered intravenously.

70. The method of claim 68, wherein the pharmaceutical composition is administered locally to a tumor.

71. The method of any one of claims 68 to 70, further comprising administering one or more additional therapeutic agents or treating the subject with one or more additional therapies.

72. The method of claim 71, wherein the treating the subject with one or more additional therapies comprises transplantation.

73. The method of claim 72, wherein the treating the subject with one or more additional therapies comprises immunotherapy.

74. The method of any one of claims 68 to 73, wherein the pharmaceutical composition is autologous to the subject.

75. The method of any one of claims 68 to 74, wherein the pharmaceutical composition is allogenic to the subject.

76. The method of any of claims 68-75, further comprising administering to the subject a pharmaceutical composition comprising a population of engineered nature killer (NK) cells.

77. The method of claim 76, wherein the engineered NK cells comprise one or more NK cells that have been modified as essentially lacking the expression of MR (killer inhibitory receptors), one or more NK cells that have been modified to express a high affinity CD16 variant, and one or more NK cells that have been modified to express one or more CARs (chimeric antigen receptors), or any combinations thereof.

78. The method of claim 77, wherein the engineered NK cells comprise one or more NK cells that have been modified as essentially lacking the expression KIR.

79. The method of claim 77, wherein the engineered NK cells comprise one or more NK cells that have been modified to express a high affinity CD16 variant.

80. The method of claim 77, wherein the engineered NK cells comprise one or more NK cells that have been modified to express one or more CARs.

81. The method of claim 77 or 80, wherein the CAR is a CAR for a tumor neo-antigen, tumor neo-epitope, HPV, PSA, PSMA, WT1, p53, MAGE-A1, MAGE-A2, MAGE-A3, MAGE-A4, MAGE-A6, MAGE-A10, MAGE-A12, BAGE, DAM-6, DAM-10, Folate receptor alpha, GAGE-1, GAGE-2, GAGE-8, GAGE-3, GAGE-4, GAGE-5, GAGE-6, GAGE-7B, NA88-A, NY-ESO-1, MART-1, MC1R, Gp100, Tyrosinase, TRP-1, TRP-2, ART-4, CAMEL, CEA, Cyp-B, HER1, HER2/neu, HERS, HER4, BRCA1, Brachyury, Brachyury (TIVS7-2, polymorphism), Brachyury (IVS7 T/C polymorphism), T Brachyury, T, hTERT, hTRT, iCE, MUC1, MUC1 (VNTR polymorphism), MUC1c, MUC1n, MUC2, PRAME, P15, RU1, RU2, SART-1, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPl/mbcr-abl, ETV6/AML, LDLR/FUT, Pml/RARα, TEL/AML1, or any combination thereof.
